SEQUENCE LISTING (3 sequences)

SEQ ID NO: 1 - DNA, 2520 bp, Homo sapiens
atgaagaaatggagcagcacagacttgggggcagctgcggacccactccaaaaggacacctgcccagaccccctggatggagaccctaactccaggccacctccagccaagccccagctctccacggccaagagccgcacccggctctttgggaagggtgactcggaggaggctttcccggtggattgccctcacgaggaaggtgagctggactcctgcccgaccatcacagtcagccctgttatcaccatccagaggccaggagacggccccaccggtgccaggctgctgtcccaggactctgtcgccgccagcaccgagaagaccctcaggctctatgatcgcaggagtatctttgaagccgttgctcagaataactgccaggatctggagagcctgctgctcttcctgcagaagagcaagaagcacctcacagacaacgagttcaaagaccctgagacagggaagacctgtctgctgaaagccatgctcaacctgcacgacggacagaacaccaccatccccctgctcctggagatcgcgcggcaaacggacagcctgaaggagcttgtcaacgccagctacacggacagctactacaagggccagacagcactgcacatcgccatcgagagacgcaacatggccctggtgaccctcctggtggagaacggagcagacgtccaggctgcggcccatggggacttctttaagaaaaccaaagggcggcctggattctacttcggtgaactgcccctgtccctggccgcgtgcaccaaccagctgggcatcgtgaagttcctgctgcagaactcctggcagacggccgacatcagcgccagggactcggtgggcaacacggtgctgcacgccctggtggaggtggccgacaacacggccgacaacacgaagtttgtgacgagcatgtacaatgagattctgatcctgggggccaaactgcacccgacgctgaagctggaggagctcaccaacaagaagggaatgatgccgctggctctggcagctgggaccgggaagatcggggtcttggcctatattctccagcgggagatccaggagcccgagtgcaggcacctgtccaggaagttcaccgagtgggcctacgggcccgtgcactcctcgctgtacgacctgtcctgcatcgacacctgcgagaagaactcggtgctggaggtgatcgcctacagcagcagcgagacccctaatcgccacgacatgctcttggtggagccgctgaaccgactcctgcaggacaagtgggacagattcgtcaagcgcatcttctacttcaacttcctggtctactgcctgtacatgatcatcttcaccatggctgcctactacaggcccgtggatggcttgcctccctttaagatggaaaaaactggagactatttccgagttactggagagatcctgtctgtgttaggaggagtctacttctttttccgagggattcagtatttcctgcagaggcggccgtcgatgaagaccctgtttgtggacagctacagtgagatgcttttctttctgcagtcactgttcatgctggccaccgtggtgctgtacttcagccacctcaaggagtatgtggcttccatggtattctccctggccttgggctggaccaacatgctctactacacccgcggtttccagcagatgggcatctatgccgtcatgatagagaagatgatcctgagagacctgtgccgtttcatgtttgtctacatcgtcttcttgttcgggttttccacagcggtggtgacgctgattgaagacgggaagaatgactccctgccgtctgagtccacgtcgcacaggtggcgggggcctgcctgcaggccccccgatagctcctacaacagcctgtactccacctgcctggagctgttcaagttcaccatcggcatgggcgacctggagttcactgagaactatgacttcaaggctgtcttcatcatcctgctgctggcctatgtaattctcacctacatcctcctgctcaacatgctcatcgccctcatgggtgagactgtcaacaagatcgcacaggagagcaagaacatctggaagctgcagagagccatcaccatcctggacacggagaagagcttccttaagtgcatgaggaaggccttccgctcaggcaagctgctgcaggtggggtacacacctgatggcaaggacgactaccggtggtgcttcagggtggacgaggtgaactggaccacctggaacaccaacgtgggcatcatcaacgaagacccgggcaactgtgagggcgtcaagcgcaccctgagcttctccctgcggtcaagcagagtttcaggcagacactggaagaactttgccctggtcccccttttaagagaggcaagtgctcgagataggcagtctgctcagcccgaggaagtttatctgcgacagttttcagggtctctgaagccagaggacgctgaggtcttcaagagtcctgccgcttccggggagaagtga

SEQ ID NO: 2 - DNA, 3360 bp, Homo sapiens
atgaagtgcagcctgaggaagatgtggcgccctggagaaaagaaggagccccagggcgttgtctatgaggatgtgccggacgacacggaggatttcaaggaatcgcttaaggtggtttttgaaggaagtgcatatggattacaaaactttaataagcaaaagaaattaaaaacatgtgacgatatggacaccttcttcttgcattatgctgcagcagaaggccaaattgagctaatggagaagatcaccagagattcctctttggaagtgctgcatgaaatggatgattatggaaatacccctctgcattgtgctgtagaaaaaaaccaaattgaaagcgttaagtttcttctcagcagaggagcaaacccaaacctccgaaacttcaacatgatggctcctctccacatagctgtgcagggcatgaataatgaggtgatgaaggtcttgcttgagcatagaactattgatgttaatttggaaggagaaaatggaaacacagctgtgatcattgcgtgcaccacaaataatagcgaagcattgcagattttgcttaacaaaggagctaagccatgtaaatcaaataaatggggatgtttccctattcaccaagctgcattttcaggttccaaagaatgcatggaaataatactaaggtttggtgaagagcatgggtacagtagacagttgcacattaactttatgaataatgggaaagccacccctctccacctggctgtgcaaaatggtgacttggaaatgatcaaaatgtgcctggacaatggtgcacaaatagacccagtggagaagggaaggtgcacagccattcattttgctgccacccagggagccactgagattgttaaactgatgatatcgtcctattctggtagcgtggatattgttaacacaaccgatggatgtcatgagaccatgcttcacagagcttcattgtttgatcaccatgagctagcagactatttaatttcagtgggagcagatattaataagatcgattctgaaggacgctctccacttatattagcaactgcttctgcatcttggaatattgtaaatttgctactctctaaaggtgcccaagtagacataaaagataattttggacgtaattttctgcatttaactgtacagcaaccttatggattaaaaaatctgcgacctgaatttatgcagatgcaacagatcaaagagctggtaatggatgaagacaacgatgggtgtactcctctacattatgcatgtagacaggggggccctggttctgtaaataacctacttggctttaatgtgtccattcattccaaaagcaaagataagaaatcacctctgcattttgcagccagttatgggcgtatcaatacctgtcagaggctcctacaagacataagtgatacgaggcttctgaatgaaggtgaccttcatggaatgactcctctccatctggcagcaaagaatggacatgataaagtagttcagcttcttctgaaaaaaggtgcattgtttctcagtgaccacaatggctggacagctttgcatcatgcgtccatgggcgggtacactcagaccatgaaggtcattcttgatactaatttgaagtgcacagatcgcttggatgaagacgggaacactgcacttcactttgctgcaagggaaggccacgccaaagccgttgcgcttcttctgagccacaatgctgacatagtcctgaacaagcagcaggcctcctttttgcaccttgcacttcacaataagaggaaggaggttgttcttacgatcatcaggagcaaaagatgggatgaatgtcttaagattttcagtcataattctccaggcaataaatgtccaattacagaaatgatagaatacctccctgaatgcatgaaggtacttttagatttctgcatgttgcattccacagaagacaagtcctgccgagactattatatcgagtataatttcaaatatcttcaatgtccattagaattcaccaaaaaaacacctacacaggatgttatatatgaaccgcttacagccctcaacgcaatggtacaaaataaccgcatagagcttctcaatcatcctgtgtgtaaagaatatttactcatgaaatggttggcttatggatttagagctcatatgatgaatttaggatcttactgtcttggtctcatacctatgaccattctcgttgtcaatataaaaccaggaatggctttcaactcaactggcatcatcaatgaaactagtgatcattcagaaatactagataccacgaattcatatctaataaaaacttgtatgattttagtgtttttatcaagtatatttgggtattgcaaagaagcggggcaaattttccaacagaaaaggaattattttatggatataagcaatgttcttgaatggattatctacacgacgggcatcatttttgtgctgcccttgtttgttgaaataccagctcatctgcagtggcaatgtggagcaattgctgtttacttctattggatgaatttcttattgtatcttcaaagatttgaaaattgtggaatttttattgttatgttggaggtaattttgaaaactttgttgaggtctacagttgtatttatcttccttcttctggcttttggactcagcttttacatcctcctgaatttacaggatcccttcagctctccattgctttctataatccagaccttcagcatgatgctaggagatatcaattatcgagagtccttcctagaaccatatctgagaaatgaattggcacatccagttctgtcctttgcacaacttgtttccttcacaatatttgtcccaattgtcctcatgaatttacttattggtttggcagttggcgacattgctgaggtccagaaacatgcatcattgaagaggatagctatgcaggtggaacttcataccagcttagagaagaagctgccactttggtttctacgcaaagtggatcagaaatccaccatcgtgtatcccaacaaacccagatctggtgggatgttattccatatattctgttttttattttgcactggggaaataagacaagaaataccaaatgctgataaatctttagaaatggaaatattaaagcagaaataccggctgaaggatcttacttttctcctggaaaaacagcatgagctcattaaactgatcattcagaagatggagatcatctctgagacagaggatgatgatagccattgttcttttcaagacaggtttaagaaagagcagatggaacaaaggaatagcagatggaatactgtgttgagagcagtcaaggcaaaaacacaccatcttgagccttag

SEQ ID NO: 3 - DNA, 3315 bp, Homo sapiens
atgtccttcgagggagccaggctcagcatgaggagccgcagaaatggtactatgggcagcacccggaccctgtactccagtgtatctcggagcacagacgtgtcctacagtgacagtgatttggtgaattttattcaggcaaattttaaaaaacgagaatgtgtcttctttaccagagactccaaggccatggagaacatatgcaagtgtggttatgcccagagccagcacatcgaaggcacccagatcaaccaaaatgagaagtggaactacaaaaaacataccaaggagtttccaacagacgccttcggggacattcagtttgagactctggggaagaaaggcaagtacttacgcttgtcctgtgacaccgactctgaaactctctacgaactgctgacccagcactggcacctcaaaacacccaacctggtcatttcagtgacgggtggagccaaaaactttgctttgaagccacgcatgcgcaagatcttcagcaggctgatttacatcgcacagtctaaaggtgcgtggattctcactggaggcactcactacggcctgatgaagtacataggcgaggtggtgagagacaacaccatcagcaggaactcagaagagaacatcgtggccattggcatcgcagcatggggcatggtctccaacagggacaccctcatcaggagctgtgatgatgagggacatttttcagctcaatacatcatggatgactttaccagagaccctctatacatcctggacaacaaccatacccacctgctgcttgtggacaacggttgtcatggacaccccacagtggaagccaagctccggaatcagctggaaaagtacatctctgagcgcaccagtcaagattccaactatggtggtaagatccccatcgtgtgttttgcccaaggaggtggaagagagactctaaaagccatcaacacctctgtcaaaagcaagatcccttgtgtggtggtggaaggctcggggcagattgctgatgtgatcgccagcctggtggaggtggaggatgttttaacctcttccatggtcaaagagaagctggtacgctttttaccacgcactgtgtcccggctgcctgaagaggaaattgagagctggatcaaatggctcaaagaaattcttgagagttctcacctactcacagtaattaagatggaagaggctggagatgagattgtgagcaacgccatttcctatgcgctgtacaaagccttcagcactaatgagcaagacaaggacaactggaatggacagctgaagcttctgctggagtggaaccagttggaccttgccagtgatgagatcttcaccaatgatcgccgctgggagtctgccgaccttcaggaggtcatgttcacggctctcataaaggacagacccaagtttgtccgcctctttctggagaatggcctgaatctgcagaagtttctcaccaatgaagtcctcacagagctcttctccacccacttcagcaccctagtgtaccggaatctgcagatcgccaagaactcctacaatgacgcactcctcacctttgtctggaagttggtggcaaacttccgtcgaagcttctggaaagaggacagaagcagcagggaggacttggatgtggaactccatgatgcatctctcaccacccggcacccgctgcaagctctcttcatctgggccattcttcagaacaagaaggaactctccaaggtcatttgggagcagaccaaaggctgtactctggcagccttgggggccagcaagcttctgaagaccctggccaaagttaagaatgatatcaacgctgctggggaatcggaggaactggccaatgaatatgagacccgagcagtggagttgttcaccgagtgttacagcaatgatgaagacttggcagaacagctactggtctactcctgcgaagcctggggtgggagcaactgtctggagctggcagtggaggctacagatcagcatttcatcgctcagcctggggtccagaatttcctttctaagcaatggtatggagagatttcccgagacacgaagaactggaagattatcctgtgtctattcatcatccccttagtgggctgtggcctcgtatcatttaggaagaaacccattgacaagcacaagaagctgctgtggtactatgtggccttcttcacgtcgcccttcgtggtcttctcctggaacgtggtcttctacatcgccttcctcctgctgtttgcctatgtgctgctcatggacttccactcagtgccacacacccccgagctgatcctctacgccctggtcttcgtcctcttctgtgatgaagtgaggcagtggtacatgaacggagtgaattatttcaccgacctatggaacgttatggacaccctgggactcttctacttcatagcgggtattgtattccggctccactcttctaataaaagctcgttgtactctgggcgcgtcattttctgtctggattacattatattcacgctaaggctcatccacattttcaccgtcagcaggaacttgggacccaagattataatgctgcagcggatgctgatcgacgttttcttcttcctgttcctctttgctgtgtggatggtggcctttggcgtggccagacaggggatcctaaggcaaaatgaacagcgctggagatggatcttccgctctgtcatctatgagccctacctggccatgtttggccaggttcccagtgacgtggatagtaccacatatgacttctcccactgtaccttctcgggaaatgagtccaagccactgtgtgtggagctggatgagcacaacctgccccgcttccctgagtggatcaccattccgctggtgtgcatctacatgctctccaccaatatccttctggtcaacctcctggtcgccatgtttggctacacggtaggcattgtacaggagaacaacgaccaggtctggaaattccagcggtacttcctggtgcaggagtactgcaaccgcctaaacatccccttccccttcgttgtcttcgcttatttctacatggtggtgaagaagtgtttcaaatgctgctgtaaagagaagaatatggagtctaatgcctgctgtttcagaaatgaggacaatgagactttggcgtgggagggtgtcatgaaggagaattaccttgtcaagatcaacacgaaagccaacgacaactcagaggagatgaggcatcggtttagacaactggactcaaagcttaacgacctcaaaagtcttctgaaagagattgctaataacatcaagtaa

(19) United States
(12) Patent Application Publication
(10) Pub. No.: US 2019/0276389 A1
(43) Pub. Date: Sep. 12, 2019

(54) SYNTHESIS OF ARYL CYCLOHEXANE ESTER DERIVATIVES USEFUL AS SENSATES IN CONSUMER PRODUCTS

(71) Applicant: The Procter & Gamble Company, Cincinnati, OH (US)

(72) Inventors: John August WOS, Mason, OH (US); Kenneth Edward YELM, Hamilton, OH (US); Gregory Mark BUNKE, Lawrenceburg, IN (US); Heath Alan FREDERICK, Harrison, OH (US); Michael REILLY, Lebanon, OH (US); John Christian HAUGHT, West Chester, OH (US); Koti Tatachar SREEKRISHNA, Mason, OH (US); Yakang LIN, Liberty Township, OH (US)

(21) Appl. No.: 15/378,547

(22) Filed: Dec. 14, 2016

Related U.S. Application Data
(60) Provisional application No. 62/269,336, filed on Dec. 18, 2015.

(51) Int. Cl. C07C 237/22; C07C 231/16; C07B 51/00; C07C 231/12; C07C 231/02

(52) U.S. Cl. CPC C07C 237/22; C07C 231/16; C07B 51/00; A61K 31/216; C07C 231/02; C07B 2200/07; C07C 231/12

(57) ABSTRACT

Personal care compositions, such as oral care and skin care compositions containing a flavor/perfume system comprising one or more coolants. The pleasant cool sensation provided by a coolant is enhanced in terms of quicker onset, greater intensity, impact or longer duration, which improves appeal and acceptability of the compositions to consumers.

### FIELD OF THE INVENTION

The present invention relates to the synthesis of cyclohexane-based ester derivatives useful as sensates. In particular the present synthetic route can be used to prepare various isomers of cyclohexane-based carboxyester coolants, such as those substituted with chiral amino acids in the alkyl bridge between the cyclohexane and aryl moieties.

### BACKGROUND OF THE INVENTION

Oral care products, such as dentifrice and mouthwash, are routinely used by consumers as part of their oral care hygiene regimens. It is well known that oral care products can provide both therapeutic and cosmetic hygiene benefits to consumers. Therapeutic benefits include caries prevention, which is typically delivered through the use of various fluoride salts; gingivitis prevention, by the use of an antimicrobial agent such as stannous fluoride, triclosan, essential oils; or hypersensitivity control through the use of ingredients such as strontium chloride or potassium nitrate. Cosmetic benefits provided by oral care products include the control of plaque and calculus formation, removal and prevention of tooth stain, tooth whitening, breath freshening, and overall improvements in mouth feel impression, which can be broadly characterized as mouth feel aesthetics. Calculus and plaque along with behavioral and environmental factors lead to formation of dental stains, significantly affecting the aesthetic appearance of teeth. Behavioral and environmental factors that contribute to teeth staining propensity include regular use of coffee, tea, cola or tobacco products, and also the use of certain oral products containing ingredients that promote staining, such as cationic antimicrobials and metal salts.

Thus daily oral care at home requires products with multiple ingredients working by different mechanisms to provide the complete range of therapeutic and aesthetic benefits, including anticaries, antimicrobial, antigingivitis, antiplaque, anticalculus and anti-erosion, as well as antiodor, mouth refreshment, stain removal, stain control and tooth whitening. In order for daily use oral care products, such as dentifrice and rinses to provide complete oral care it is often necessary to combine actives and additives, many of which have the disadvantage of causing negative aesthetics during use, in particular unpleasant taste and sensations and stain promotion. The unpleasant taste and mouth sensations have been described as having one or more of bitter, metallic, astringent, salty, numbing, stinging, burning, or prickling, and even irritating aspects. Typical ingredients for oral care use that are associated with these aesthetic negatives include antimicrobial agents such as cetyl pyridinium chloride, chlorhexidine, stannous and zinc salts; tooth bleaching agents such as peroxides; antitartar agents such as pyrophosphate, tripolyphosphate and hexametaphosphate; and excipients, such as baking soda and surfactants. To mitigate the aesthetic negatives from these ingredients, oral care products are typically formulated with flavoring agents, sweeteners and coolants to taste as good as possible and provide a pleasant experience. In particular, it is desirable for oral care products to provide a refreshing cooling sensation during and after use. In addition to mitigation of negative sensations, sensate molecules are formulated into oral care compositions to convey a signal of efficacy. Such signals of efficacy include cooling, tingling, numbing, warming, sweetness, and rheological sensations such as phase change and fizzing or bubbling.

A large number of coolant compounds of natural or synthetic origin have been described. The most well-known compound is menthol, particularly 1-menthol, which is found naturally in peppermint oil, notably of Mentha arvensis L and Mentha viridis L. Of the menthol isomers, the 1-isomer occurs most widely in nature and is typically what is referred by the name menthol having coolant properties. L-menthol has the characteristic peppermint odor, has a clean fresh taste and exerts a cooling sensation when applied to the skin and mucosal surfaces. Other isomers of menthol (neomenthol, isomenthol and neoisomenthol) have somewhat similar, but not identical odor and taste, i.e., some having disagreeable notes described as earthy, camphor, musty. The principal difference among the isomers is in their cooling potency. L-menthol provides the most potent cooling, i.e., having the lowest cooling threshold of about 800 ppb, i.e., the concentration where the cooling effect could be clearly recognized. At this level, there is no cooling effect for the other isomers. For example, d-neomenthol is reported to have a cooling threshold of about 25,000 ppb and 1-neomenthol about 3,000 ppb. (R. Emberger and R. Hopp, “Synthesis and Sensory Characterization of Menthol Enantiomers and Their Derivatives for the Use in Nature Identical Peppermint Oils,” Specialty Chemicals (1987), 7(3), 193-201). This study demonstrated the outstanding sensory properties of 1-menthol in terms of cooling and freshness and the influence of stereochemistry on the activity of these molecules.

Among synthetic coolants, many are derivatives of or are structurally related to menthol, i.e., containing the cyclohexane moiety, and derivatized with functional groups including carboxamide, ketal, ester, ether and alcohol. Examples include the ρ-menthanecarboxamide compounds, such as N-ethyl-ρ-menthan-3-carboxamide, known commercially as “WS-3”, and others in the series, such as WS-5 (N-ethoxycarbonylmethyl-ρ-menthan-3-carboxamide), WS-12 [N-(4-methoxyphenyl)-ρ-menthan-3-carboxamide] and WS-14 (N-tert-butyl-ρ-menthan-3-carboxamide). Examples of menthane carboxy esters include WS-4 and WS-30. An example of a synthetic carboxamide coolant that is structurally unrelated to menthol is N,2,3-trimethyl-2-isopropylbutanamide, known as “WS-23”. Additional examples of synthetic coolants include alcohol derivatives such as 3-(1-menthoxy)-propane-1,2-diol known as TK-10, isopulegol (under the tradename Coolact P) and ρ-menthane-3,8-diol (under the tradename Coolact 38D); menthone glycerol acetal known as MGA; menthyl esters such as menthyl acetate, menthyl acetoacetate, menthyl lactate known as Frescolat* supplied by Haarmann and Reimer, and monomenthyl succinate under the tradename Physcool from V. Mane. TK-10 is described in U.S. Pat. No. 4,459,425. Other alcohol and ether derivatives of menthol are described e.g., in GB 1,315,626 and in U.S. Pat. Nos. 4,029,759; 5,608,119; and 6,956,139. WS-3 and other carboxamide cooling agents are described for example in U.S. Pat. Nos. 4,136,163; 4,150,052; 4,153,679; 4,157,384; 4,178,459 and 4,230,688. Additional N-substituted ρ-menthane carboxamides are described in WO 2005/049553A1 including N-(4-cyanomethylphenyl)-ρ-menthanecarboxamide, N-(4-sulfamoylphenyl)-ρ-menthanecarboxamide, N-(4-cyanophenyl)-ρ-menthanecarboxamide, N-(4-acetylphenyl)-ρ-menthanecarboxamide, N-(4-hydroxymethylphenyl)-ρ-menthanecarboxamide and N-(3-hydroxy-4-methoxyphenyl)-ρ-menthanecarboxamide Other N-substituted ρ-menthane carboxamides include amino acid derivatives such as those disclosed in WO 2006/103401 and in U.S. Pat. Nos. 4,136,163; 4,178,459 and 7,189,760 such as N-((5-methyl-2-(1-methylethyl)cyclohexyl)carbonyl)glycine ethyl ester and N-((5-methyl-2-(1-methylethyl)cyclohexyl)carbonyl)alanine ethyl ester. Menthyl esters, including those of amino acids such as glycine and alanine are disclosed e.g., in EP 310 299 and in U.S. Pat. Nos. 3,111,127; 3,917,613; 3,991,178; 5,703,123; 5,725,865; 5,843,466; 6,365,215; 6,451,844; and 6,884,903. Ketal derivatives are described, e.g., in U.S. Pat. Nos. 5,266,592; 5,977,166 and 5,451,404. Additional agents that are structurally unrelated to menthol but have been reported to have a similar physiological cooling effect include alpha-keto enamine derivatives described in U.S. Pat. No. 6,592,884 including 3-methyl-2-(1-pyrrolidinyl)-2-cyclopenten-1-one (3-MPC), 5-methyl-2-(1-pyrrolidinyl)-2-cyclopenten-1-one (5-MPC), and 2,5-dimethyl-4-(1-pyrrolidinyl)-3(2H)-furanone (DMPF); icilin (also known as AG-3-5, chemical name 1-[2-hydroxyphenyl]-4-[2-nitrophenyl]-1,2,3,6-tetrahydropyrimidine-2-one) described in Wei et al., J. Pharm. Pharmacol. (1983), 35:110-112. Reviews on the coolant activity of menthol and synthetic coolants include H. R. Watson, et al. J. Soc. Cosmet. Chem. (1978), 29, 185-200 and R. Eccles, J. Pharm. Pharmacol., (1994), 46, 618-630.

Molecules with chiral centers can drive different biological responses depending upon the spatial orientation of specific moieties on those molecules. The biological responses tend to differ where these molecules interact with a receptor. In the flavor and fragrance realm, a well-known example of such chiral diversity is Carvone. The R-(−) enantiomers of Carvone connotes a spearmint taste and scent, where the S-(+) enantiomer has a taste and smell like caraway seeds. Limonene is another molecule where the spatial orientation of the chiral center affects its scent. For example, the R-(+) isomer of limonene has a citrus scent, where the S-(−) isomer smells like turpentine. For synthetic molecules, the ability to control the stereochemistry during the synthesis steps, gives the ability to select for the finished molecule with the desired sensorial properties. An object of this invention is a method of synthesis to control the stereochemistry of amino acid substituted cyclohexane carboxyesters.

The present invention provides one or more coolants and methods of synthesis, wherein the cooling and refreshing sensation provided by the coolants is improved over current coolants in terms of onset, intensity, and/or duration.

### SUMMARY OF THE INVENTION

A compound is provided that comprises the following structure:

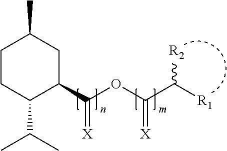

n=0 to 4; m=0 to 7; and wherein X is independently selected from O or H,H

R₁ and R₂ are independently selected from H, C, alkyl, aryl, substituted aryl, heteroalkyl, amino, amido, aminoalkyl, alkoxy; or when bound together, form part of an aromatic ring system.

A compound is provided having the structure shown above, wherein the compound at a concentration of about 5.2E-5% provides a greater activation of TRPM8 than WS5 at a concentration of about 30 mM; a greater activation of TRPA1 than allyl isothiocyanate at a concentration of about 50 mM; and a greater activation of TRPV1 than capsaicin at a concentration of about 350 nM.

A compound having the structure shown above is provided, wherein the compound at a concentration of about 5.2E-5% provides at least about 100%, 105%, 110%, 115%, 120% 125% or 130% activation of TRPM8 when compared to WS5 at a concentration of about 30 mM; at least about 100%, 130%, 140%, 150%, 160%, 170%, 180%, 190%, 200%, 210%, 220%, 230% or 240% activation of TRPA1 when compared to allyl isothiocyanate at a concentration of about 50 mM; and at least about 95%, 100%, 105%, 110%, or 115% activation of TRPV1 when compared to capsaicin at a concentration of about 350 nM.

A compound is provided that comprises the following structure:

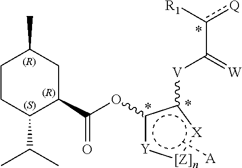

R₁ is selected from H, alkyl, amino alkyl, alkoxy

Q=H₂, O, OR₁, N(R₁)₂

V=NR₁, O

W=H₂, O

X, Y=independently selected from H, aryl, naphthyl for n=0

X, Y=aliphatic CH₂ or aromatic CH for n≥1 and Z is selected from aliphatic CH₂, aromatic CH, or heteroatom

A=lower alkoxy, lower alkylthio, aryl, substituted aryl or fused aryl

and stereochemistry is variable at the positions marked*

A compound is provided that comprises the following structure:

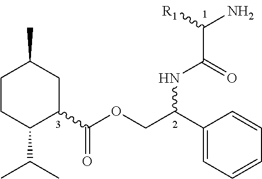

R₁ is selected from H, alkyl, amino alkyl, alkoxy;

and wherein in certain embodiments the variable stereochemistry at position #1, where the variable stereochemistry at position #1, when R1 is an alkyl group, is either L or D; the stereochemistry position at position #2 is in the S-position; and the stereochemistry from the menthyl moiety at position #3 is in the L or in the neo-configuration.

A personal care composition is provided that comprises a compound having the following structure:

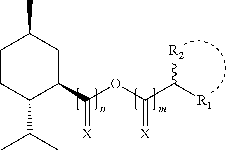

n=0 to 4; m=0 to 7; and wherein X is independently selected from O or H,H

R₁ and R₂ are independently selected from H, C, alkyl, aryl, substituted aryl, heteroalkyl, amino, amido, aminoalkyl, alkoxy; or when bound together, form part of an aromatic ring system; and wherein the compound activates at least one of TRPV1, TRPA1, or TRPM8.

A personal care composition is provided that comprises a compound having the following structure:

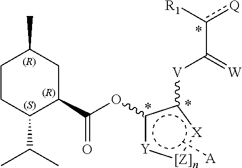

R₁ is selected from H, alkyl, amino alkyl, alkoxy

Q=H₂, O, OR₁, N(R₁)₂

V=NR₁, O

W=H₂, O

X, Y=independently selected from H, aryl, naphthyl for n=0

X, Y=aliphatic CH₂ or aromatic CH for n≥1 and Z is selected from aliphatic CH₂, aromatic CH, or heteroatom

A=lower alkoxy, lower alkylthio, aryl, substituted aryl or fused aryl

and stereochemistry is variable at the positions marked*.

A method of preparing esters of menthol and menthol derivatives of Formula (I) is provided:

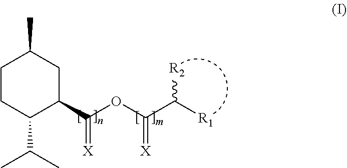

n=0 to 4; m=0 to 7; and wherein X is independently selected from O or H,H

R₁ and R₂ are independently selected from H, C, alkyl, aryl, substituted aryl, heteroalkyl, amino, amido, aminoalkyl, alkoxy; or when bound together, form part of an aromatic ring system.

comprising the steps of reacting an alcohol of Formula (II) in a coupling reaction:

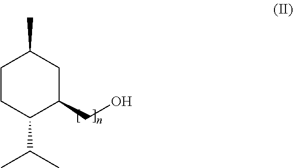

wherein n=0 to 4;

with an activated carboxylic acid derivative of Formula (III):

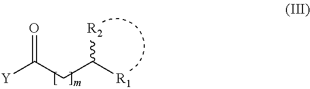

and wherein m=0 to 6 and Y is an activated leaving group.

A method of preparing menthylcarboxylic acid ester derivatives of Formula (I) is provided:

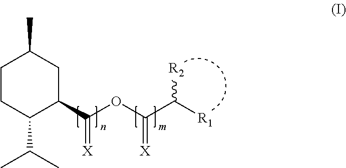

n=0 to 4; m=0 to 7; and wherein X is independently selected from O or H,H

R₁ and R₂ are independently selected from H, C, alkyl, aryl, substituted aryl, heteroalkyl, amino, amido, aminoalkyl, alkoxy; or when bound together, form part of an aromatic ring system.

comprising the steps of reacting a carboxylic acid derivative of Formula (II):

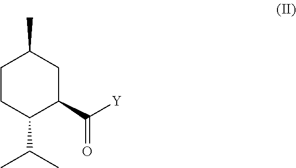

wherein Y is an activated leaving group;

in a coupling reaction with an alcohol derivative of Formula (III):

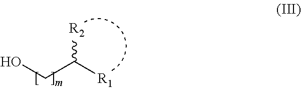

and wherein m=1 to 6.

The present invention provides a synthetic route for preparing selected isomers of amino acid substituted cyclohexane substituted carboxy esters, in particular those substituted with an amino acid at the amine linking the aryl moiety to the cyclohexane carboxy ester.

The invention provides for compositions containing the S-isomer of amino acid substituted aryl cyclohexane carboxy esters, which provide long lasting sensorial stimulations, such as cooling, warming, tingling, numbing, and pain mitigation.

In certain embodiments all compounds disclosed in this specification also include any acceptable salts or solvates thereof.

### DETAILED DESCRIPTION OF THE INVENTION

The present invention outlines a series of menthol esters and methods of synthesizing menthol esters built off of an (S)-2-phenyl-amino acid backbone or an (R)-2-phenyl-amino acid backbone, depending upon the desired diastereomer of the end product. Where the amino acid can be in the D or L form and may be natural or unnatural. Examples of amino acids that can be used include (D)-alanine, (L)-alanine, or glycine. These molecules have low EC50 values on TRPM8, TRPA1, and TRPV1 and drive a neural stimulated cooling response.

A second aspect of the invention is the moving of the ester carbonyl linkage off the menthol moiety so that the ester comes from the alkyl group linked to the cyclohexane moiety via the O—C(O) linkage, where the (S)-2-phenyl-amino acid backbone or an (R)-2-phenyl-amino acid backbone was added off the alkyl ester. Examples of amino acids that may be used include (D)-alanine, (L)-alanine, or glycine. These molecules have low EC50 values on TRPM8, TRPA1, and TRPV1 and drive a neural stimulated cooling response.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at 25° C. unless otherwise specified.

By “personal care composition” is meant a product, which in the ordinary course of usage is applied to or contacted with a body surface to provide a beneficial effect. Body surface includes skin, for example dermal or mucosal; body surface also includes structures associated with the body surface for example hair, teeth, or nails. Examples of personal care compositions include a product applied to a human body for improving appearance, cleansing, and odor control or general aesthetics. Non-limiting examples of personal care compositions include oral care compositions, such as, dentifrice, mouth rinse, mousse, foam, mouth spray, lozenge, chewable tablet, chewing gum, tooth whitening strips, floss and floss coatings, breath freshening dissolvable strips, denture care product, denture adhesive product; after shave gels and creams, pre-shave preparations, shaving gels, creams, or foams, moisturizers and lotions; cough and cold compositions, liquids, gels, gel caps, tablets, and throat sprays; leave-on skin lotions and creams, shampoos, body washes, body rubs, such as Vicks Vaporub; hair conditioners, hair dyeing and bleaching compositions, mousses, shower gels, bar soaps, antiperspirants, deodorants, depilatories, lipsticks, foundations, mascara, sunless tanners and sunscreen lotions; feminine care compositions, such as lotions and lotion compositions directed towards absorbent articles; baby care compositions directed towards absorbent or disposable articles; and oral cleaning compositions for animals, such as dogs and cats.

The term “dentifrice”, as used herein, includes tooth or subgingival—paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

The term “dispenser”, as used herein, means any pump, tube, or container suitable for dispensing compositions such as dentifrices.

The term “teeth”, as used herein, refers to natural teeth as well as artificial teeth or dental prosthesis.

The term “orally acceptable carrier or excipients” includes safe and effective materials and conventional additives used in oral care compositions including but not limited to fluoride ion sources, anti-calculus or anti-tartar agents, buffers, abrasives such as silica, alkali metal bicarbonate salts, thickening materials, humectants, water, surfactants, titanium dioxide, flavorants, sweetening agents, xylitol, coloring agents, and mixtures thereof.

Herein, the terms “tartar” and “calculus” are used interchangeably and refer to mineralized dental plaque biofilms.

The present invention is also directed towards “oral health compositions” as used herein refers to compositions in a form that is deliverable to a mammal in need via the oral cavity, mouth, throat, nasal passage or combinations thereof. Nonlimiting examples include liquid compositions, cough syrups, respiratory preparations, beverage, supplemental water, pills, soft gels, tablets, capsules, gel compositions and foam compositions, saline wash and combinations thereof. Liquid compositions, gel compositions can be in a form that is directly deliverable to the mouth and throat. These compositions can be delivered by a delivery device selected from droppers, pump, sprayers, liquid dropper, saline wash delivered via nasal passageway, cup, bottle, liquid filled gel, liquid filled gummy, center filled gum, chews, films, center filled lozenge, gum filled lozenge, pressurized sprayers, atomizers, air inhalation devices, liquid filled compressed tablet, liquid filled gelatin capsule, liquid filled capsule, squeezable sachets, power shots, and other packaging and equipment, and combinations thereof. The sprayer, atomizer, and air inhalation devices can be associated with a battery or electric power source.

The present invention is also directed towards a respiratory preparation. In one embodiment the respiratory preparation comprises a film forming agent and a thickening agent; and provides on demand relief. The preparation can work to physically coat the mouth and throat creating a soothing barrier over the epithelial cells that line the throat layer. The preparation can additionally, reduce inflammation and relieve minor pain associated with a cough or sore throat. In certain embodiments the respiratory preparation would not contain a pharmaceutical active.

The present invention is also directed to lotion compositions and to absorbent articles, particularly disposable absorbent articles, having a lotion treatment composition applied thereon. Disposable absorbent articles can be baby diapers or feminine hygiene articles, including incontinence devices and catamenial products, such as tampons, sanitary napkins, pantiliners, interlabial products, and the like.

The absorbent article can comprise any known or otherwise effective topsheet, such as one which is compliant, soft feeling, and non-irritating to the body of the wearer. Suitable topsheet materials include a liquid pervious material that is oriented towards and contacts the body of the wearer, thereby permitting body discharges to rapidly penetrate through the topsheet without allowing fluid to flow back through the topsheet to the skin of the wearer. The topsheet, while capable of allowing rapid transfer of fluid through it, also provides for the transfer or migration of the lotion composition onto an external or internal portion of a body of the wearer. A suitable topsheet can be made of various materials, such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof, as is well known in the art of making catamenial products such as sanitary napkins, pantiliners, incontinence pads, and the like.

Components of the present compositions are described in the following paragraphs.

| SEQ ID NO | Sequence |
|---|---|
| 1 | Human TRPV1 DNA sequence |
| 2 | Human TRPA1 DNA sequence |
| 3 | Human TRPM8 DNA sequence |

A sequence listing that sets forth the nucleotide sequences for SEQ ID NO: 1 to 3 herein is being filed concurrently with the present application as an ASCII text file titled “14174M_Nucleotide_Sequence_Listing_ST25.” The ASCII text file was created on 13 Dec. 2016 and is 13 Kbytes in size. In accordance with MPEP § 605.08 and 37 CFR § 1.52(e), the subject matter in the ASCII text file is incorporated herein by reference.

The term “TRPV1” or “TRPV1 receptor”, as used herein, refers to the transient receptor potential vanilloid receptor 1, which is a ligand-gated, non-selective cation channel preferentially expressed on small-diameter sensory neurons and detects noxious as well as other substances. The TRPV1 receptor is provided as SEQ ID NO: 1. The TRPV1 receptor responds to, for example, both noxious and painful stimuli. A noxious stimulus would include those that give a burning (i.e. hot) sensation.

The term “TRPV1 agonist”, as used herein, refers to any compound, which at a concentration of 1 mM gives a calcium flux count of at least 1000 counts or 20% above the background level of calcium present in the cell according to the FLIPR method, as discussed herein. The term “count” is defined as the change in fluorescence of the cell lines due to the influx of calcium across the cell membrane, which reacts with the calcium sensitive dye present within the cells.

The term “TRPV1 enhancer”, as used herein, refers to any compound that boosts the calcium flux activity of an agonist that directly activates TRPV1, but does not directly activate TRPV1.

The term “TRPV1 antagonist”, as used herein, refers to any component which at a concentration of 1 mM gives a reduction in calcium flux count of at least 1000 counts or 20% below the activation of TRPV1 receptor by 100 mM of hydrogen peroxide or 100 mM L-menthol of calcium present in the cell according to the FLIPR method, as discussed herein. The term “count” is defined as the change in fluorescence of the cell lines due to the influx of calcium across the cell membrane, which reacts with the calcium sensitive dye present within the cells. The antagonistic effect may also be measured by looking at lower concentrations of the receptor agonist, such as hydrogen peroxide or L-menthol at 500 μM or lower. In certain embodiments a TRPV1 receptor antagonist at a concentration of greater than 100 mM does not give a reduction of at least 20% below the maximum calcium flux count from the TRPV1 receptor activated by 350 μM capsaicin.

The term “TRPA1” or “TRPA1 receptor”, as used herein, refers to the transient receptor potential cation channel, subfamily A, member 1, having a large cysteine-rich N-terminus that contains 18 predicted ankyrin repeats. The TRPA1 receptor is provided as SEQ ID NO: 2. TRPA1 is a ligand-gated, non-selective cation channel preferentially expressed on small diameter sensory neurons.

The term “TRPA1 agonist”, as used herein, refers to any compound, which at a concentration of 1 mM gives a calcium flux count of at least 1000 counts or 20% above the background level of calcium present in the cell according to the FLIPR method, as discussed herein. The term “count” is defined as the change in fluorescence of the cell lines due to the influx of calcium across the cell membrane, which reacts with the calcium sensitive dye present within the cells.

The term “TRPA1 enhancer”, as used herein, refers to any compound that boosts the calcium flux activity of an agonist that directly activates TRPA1, but does not directly activate TRPA1.

The term “TRPA1 antagonist”, as used herein, refers to any component, which at a concentration of 1 mM gives a reduction in calcium flux count of at least 1000 counts or 20% below the activation of TRPA1 receptor by 100 mM of hydrogen peroxide or 100 mM L-menthol of calcium present in the cell according to the FLIPR method, as discussed herein. The term “count” is defined as the change in fluorescence of the cell lines due to the influx of calcium across the cell membrane, which reacts with the calcium sensitive dye present within the cells. The antagonistic effect may also be measured by looking at lower concentrations of the receptor agonist, such as hydrogen peroxide or L-menthol at 100 μM or lower. In certain embodiments a TRPA1 receptor antagonist at a concentration of greater than 100 mM does not give a reduction of at least 20% below the maximum calcium flux count from the TRPA1 receptor activated by 50 mM allyl isothiocyanate.

The cooling receptor conventionally known as TRPM8 or the menthol receptor has been demonstrated as a means to differentiate intensity and duration of organic molecules that initiate and propagate the non-thermal cooling perception (D. D. Mckemy, The Open Drug Discovery Journal 2:81-88 2010). McKemy reported the EC50 values of many agonists to TRPM8 which span the range of 100 nM to 19 mM, thus showing the channel can be activated across a wide range of structures at varying concentrations. This channel also has the nomenclature of CRM1 and TRPP8. The later was designated as such due to its identification with prostate cells, where it was employed as a means to identify molecules targeted towards prostate cancer.

The term “TRPM8” or “TRPM8 receptor”, as used herein, refers to cold- and menthol-sensitive receptor (CMR1) or TRPM8. The TRPM8 nomenclature for the receptor comes from its characterization as a non-selective cation channel of the transient receptor potential (TRP) family that is activated by stimuli including low temperatures, menthol and other chemical coolants. The TRPM8 receptor is provided as SEQ ID NO: 3.

The term “TRPM8 agonist”, as used herein, refers to any compound, which when added to a TRPM8 receptor, according to the FLIPR method, as discussed herein, produces any increase in fluorescence over background.

The term “TRPM8 enhancer”, as used herein, refers to any compound that boosts the calcium flux activity of an agonist that directly activates TRPM8, but does not directly activate TRPM8.

The term “TRPM8 antagonist”, as used herein, refers to any compound, which does not show any agonistic activity when directly added and inhibits activation of the TRPM8 receptor by a known TRPM8 agonist. Using the FLIPR method, as discussed herein a molecule that has >20% reduction in calcium flux compared to the WS5 activated TRPM8 receptor is considered a TRPM8 antagonist.

The term potency, as defined by the Merck Manual, refers to the concentration (EC50) or dose (ED50) of a chemistry required to produce 50% of the chemistry's maximal effect as depicted by a graded dose-response curve. EC50 equals Kd (Dissociation constant, which is a measure of 50% of the substance in question bound to the receptor) when there is a linear relationship between occupancy and response. Often, signal amplification occurs between receptor occupancy and response, which results in the EC50 for response being much less (ie, positioned to the left on the abscissa of the log dose-response curve) than KD for receptor occupancy. Potency depends on both the affinity of chemistry for its receptor, and the efficiency with which chemistry-receptor interaction is coupled to response. The dose of chemistry required to produce an effect is inversely related to potency. In general, low potency is important only if it results in a need to administer the chemistry in large doses that are impractical. Quantal dose-response curves provide information on the potency of chemistry that is different from the information derived from graded dose-response curves. In a quantal dose-response relationship, the ED50 is the dose at which 50% of individuals exhibit the specified quantal effect.

Coolants or compounds that have a physiological cooling effect particularly on oral and other mucosal surfaces and skin are common ingredients in a wide variety of products, including edible compositions, personal care compositions, and in flavor or perfume compositions. Examples of edible compositions include confectionery, candies, chocolate, chewing gum, beverages and oral medicines. Personal care compositions, including oral care compositions, have been described previously. The pleasant cooling sensation provided by coolants contributes to the appeal and acceptability of the products. In particular, oral care products, such as dentifrices and mouthwashes are formulated with coolants because they provide breath freshening effects and a clean, cool, fresh feeling in the mouth.

Unlike traditional carboxamide coolants, the cyclohexane carboxy esters of the present invention were built off of a (S)-2-phenyl glycine backbone or an (R)-2-phenyl glycine backbone, depending upon the desired diastereomer at position 2 in the structure below.

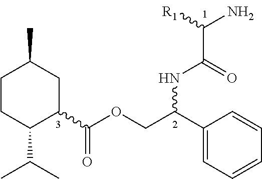

where the variable stereochemistry at position #1, where the variable stereochemistry at position #1, when R1 is an alkyl group, is either L or D; the stereochemistry position at position #2 is in the S-configuration; and the stereochemistry from the menthyl moiety at position #3 is in the L or in the neo-configuration.

### General Description for Synthesis of Cyclohexyl Carboxylate Ester Derivatives (Scheme 1)

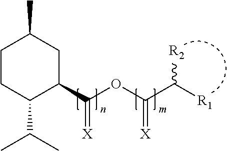

n=0 to 4; m=0 to 7; and wherein X is independently selected from O or H,H

R₁ and R₂ are independently selected from H, C, alkyl, aryl, substituted aryl, heteroalkyl, amino, amido, aminoalkyl, alkoxy; or when bound together, form part of an aromatic ring system.

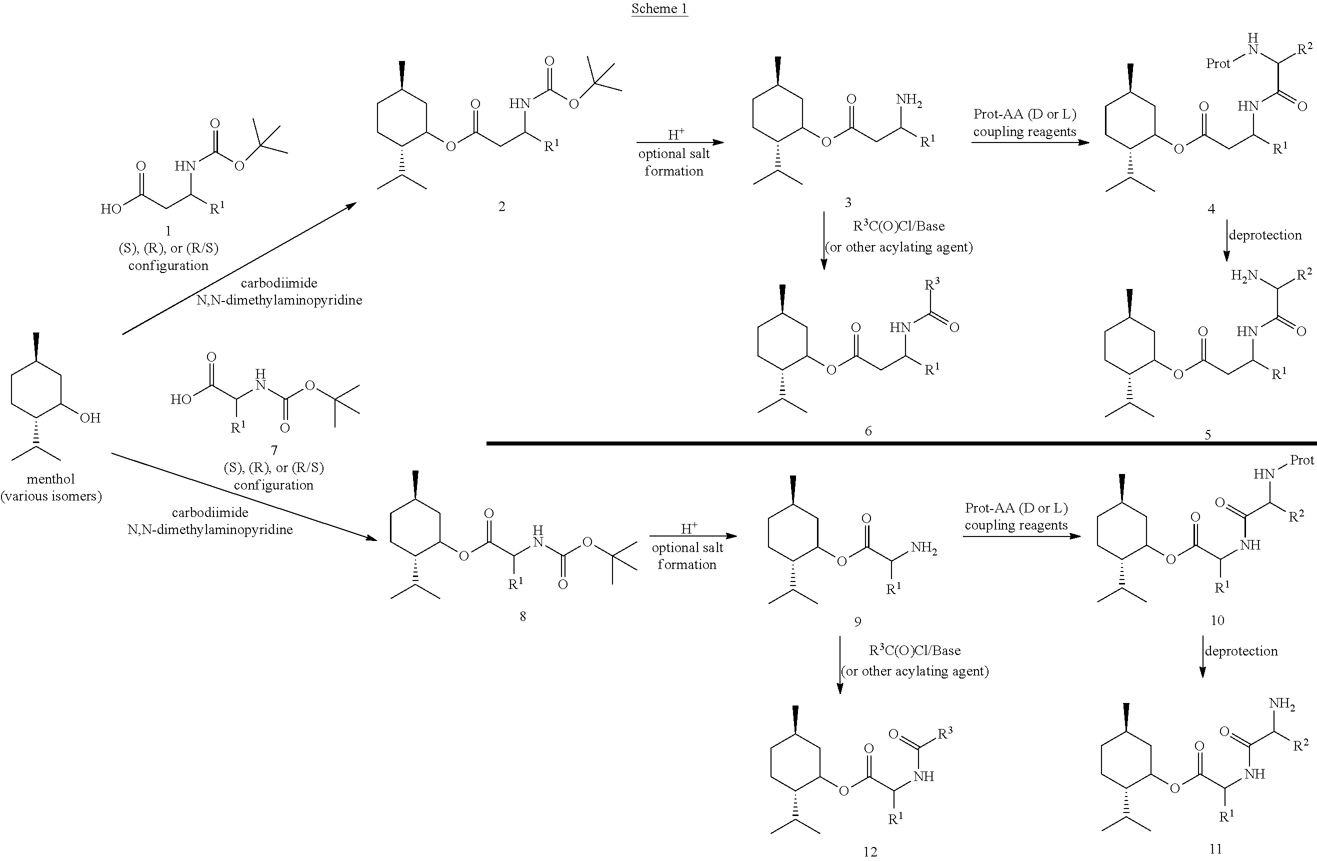

In general, cyclohexyl carboxylate ester analogs described can be synthesized by the routes described in Scheme 1. In a general description, menthol or another substituted cyclohexanol can be esterified with N-protected β-amino acids (1) by methods common in the art (US Pub. No. 2012/0028995) to provide protected amino esters (2). The protected amino esters (2) can be deprotected to amino esters (3) which can subsequently be capped via acylation with activated carboxylic acids (acid chlorides, anhydrides, etc.) to provide N-substituted esters (6). The amino esters (3) can also be capped via acylation with protected amino acids to provide a variety of N-substituted cyclohexyl esters (4) which can be further manipulated via deprotection or tested for TRPM8, TRPA1, and TRPV1 activity as independent chemical entities. Upon deprotection of these materials (4) the cyclohexyl esters (5) are produced.

Closely related cyclohexyl carboxylate ester analogs derived from α-amino acids can also be synthesized using a route similar to that described in Scheme 1. In a general description, menthol or another substituted cyclohexanol can be esterified with N-protected α-amino acids (7) by methods common in the art (U.S. Pat. No. 8,637,547) to provide protected amino esters (8). The protected amino esters (8) can be deprotected to amino esters (9) which can subsequently be capped via acylation with activated carboxylic acids (acid chlorides, anhydrides, etc.) to provide N-substituted esters (12). The amino esters (9) can also be capped via acylation with protected amino acids to provide a variety of N-substituted cyclohexyl esters (10) which can be further manipulated via deprotection or tested for TRPM8, TRPA1, and TRPV1 activity as independent chemical entities. Upon deprotection of these materials (10) the cyclohexyl esters (11) are produced.

### General Description for Synthesis of Cyclohexane Carboxy Ester Derivatives (Scheme 2)

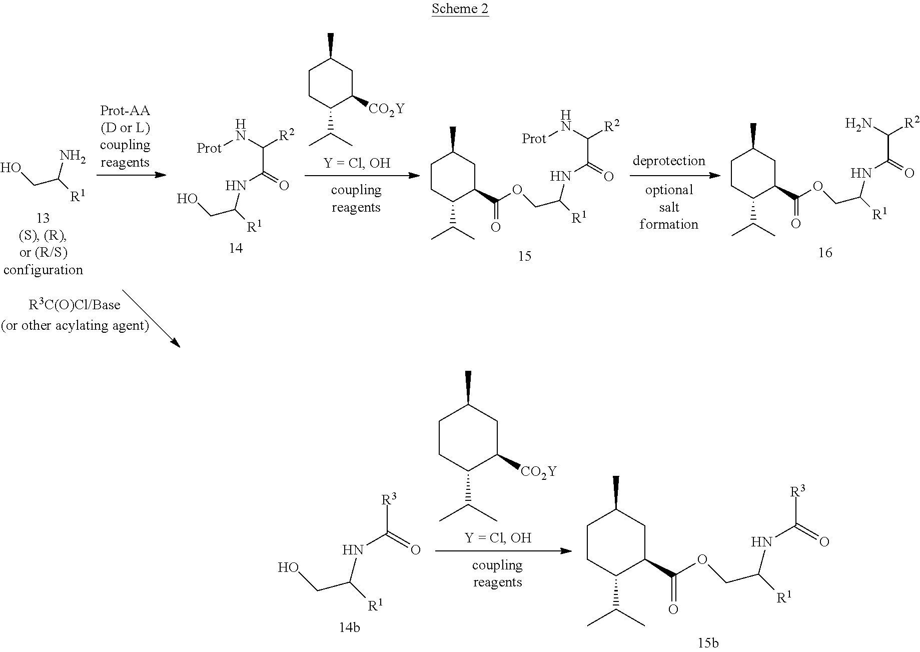

In general, cyclohexane carboxy ester analogs described can be synthesized by the routes described in Scheme 2. The aminoalcohol (13) can be coupled to an appropriately protected α-aminoacid derivative to provide the amide derivative (14). Esterification of (14) with an activated cyclohexanecarboxylic acid provides a variety of N-substituted cyclohexyl esters (15) which can be further manipulated via deprotection or tested for TRPM8, TRPA1, and TRPV1 activity as independent chemical entities. Upon deprotection of these materials (15) the cyclohexane carboxy esters (16) are produced. The aminoalcohol (13) can also be acylated with an acid chloride or other acylating agent to provide the amide derivative (14b). Esterification of (14b) with an activated cyclohexanecarboxylic acid provides a variety of N-substituted cyclohexyl esters (15b) which can be tested for TRPM8, TRPA1, and TRPV1 activity.

### General Description for Synthesis of Cyclohexylmethyl Carboxylate Derivatives (Scheme 3)

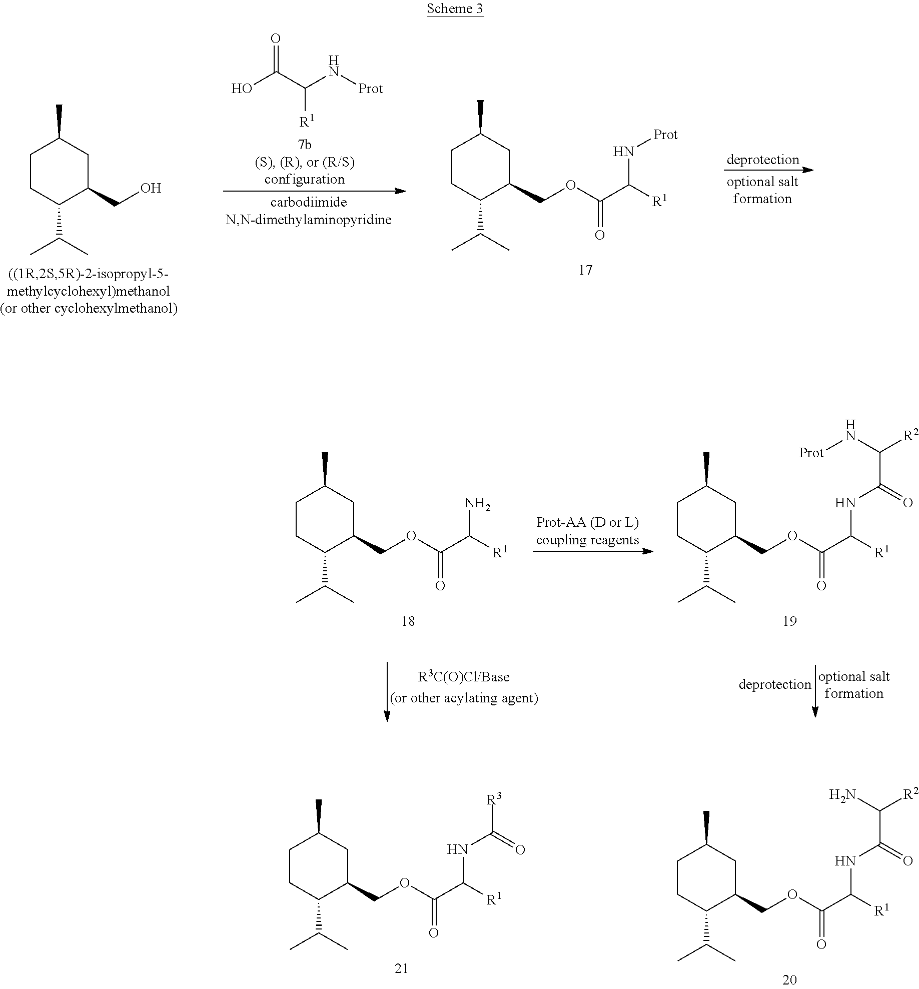

In general, cyclohexylmethyl carboxylate analogs, as previously described, can be synthesized by the routes described in Scheme 3. ((1R,2S,5R)-2-isopropyl-5-methylcyclohexyl)methanol or other cyclohexylmethanol can be obtained commercially or prepared by methods common in the art (E.g., Yang, Zhang, Ouyang; Xiangtan Daxue Ziran Kexue Xuebao (2009), 31(3), 96-100 and Kato, Ueda, Hashimoto; Agricultural and Biological Chemistry (1970), 34(1), 28-31). In a general description ((1R,2S,5R)-2-isopropyl-5-methylcyclohexyl)methanol or other cyclohexylmethanol can be esterified with N-protected α-amino acids (7b) by methods common in the art (U.S. Pat. No. 8,637,547) to provide protected amino esters (17). The protected amino esters (17) can be deprotected to amino esters (18) which can subsequently be capped via acylation with activated carboxylic acids (acid chlorides, anhydrides, etc.) to provide N-substituted esters (21). The amino esters (18) can also be capped via acylation with protected amino acids to provide a variety of N-substituted cyclohexyl esters (19) which can be further manipulated via deprotection or tested for TRPM8, TRPA1, and TRPV1 activity as independent chemical entities. Upon deprotection of these materials (19) the cyclohexylmethyl esters (20) are produced.

### General Description for Synthesis of Cyclohexyl Anthranilate Derivatives (Scheme 4)

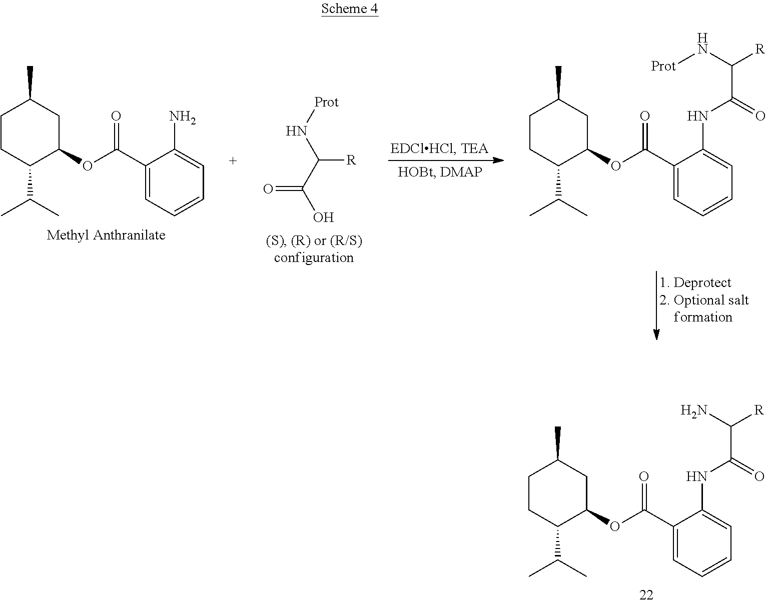

In general, cyclohexyl anthranilate derivatives can be coupled with N-protected α-amino acids by methods common in the art (U.S. Pat. No. 8,637,547) plus the addition of 4-(dimethylamino)pyridine to provide protected amino esters. The protected amino esters can be deprotected to amino esters (22).

Other omega-amino cyclohexane esters can be envisioned that provide a six to eight atom spacing between the cyclohexane ring a terminal basic amino group. These are illustrated in Scheme 5-7.

### General Description for Synthesis of Omega-Amino Cyclohexyl Carboxylate Analogs (Scheme 5)

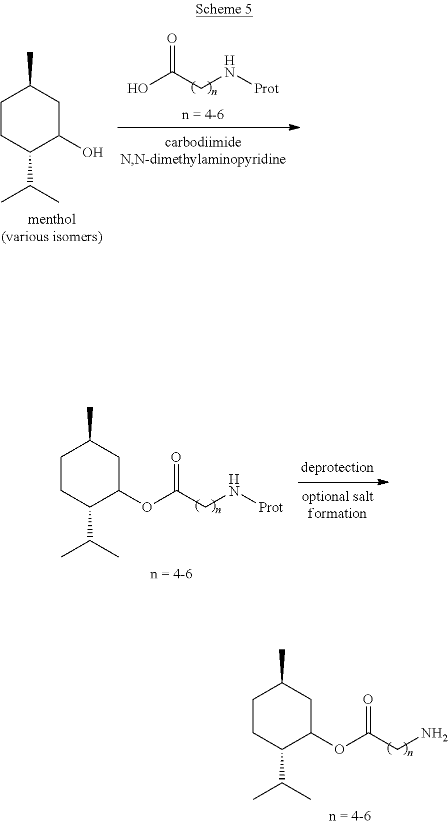

In general, omega-amino cyclohexyl carboxylate ester analogs described can be synthesized by the route described in Scheme 5. In a general description, menthol or another substituted cyclohexanol can be esterified with N-protected ω-amino acids by methods common in the art (US Pub. No. 2012/0028995) to provide protected amino esters. The protected amino esters can be deprotected to provide omega-amino cyclohexyl carboxylate ester.

### General Description for Synthesis of Omega-Amino Cyclohexane Carboxy Ester Derivatives (Scheme 6)

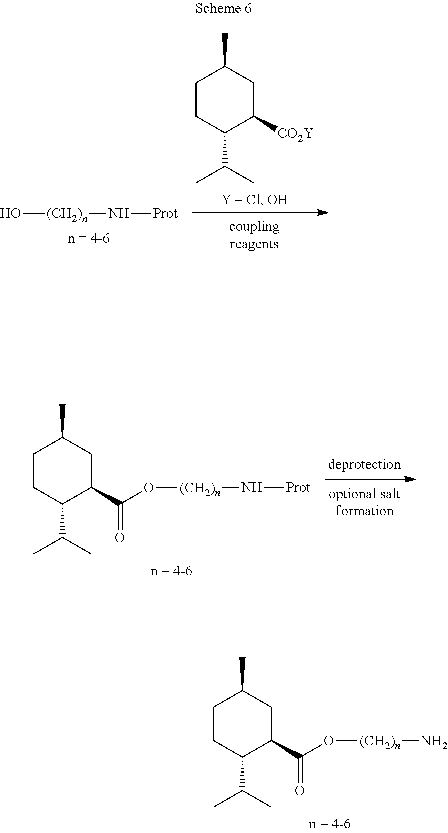

In general, omega-amino cyclohexane carboxy ester analogs can be synthesized by the route described in Scheme 6. Esterification of an N-protected ω-aminoalcohol with an activated cyclohexanecarboxylic acid can provide a variety of N-protected esters which can be further manipulated via deprotection or tested for TRPM8, TRPA1, and TRPV1 activity as independent chemical entities. Upon deprotection of these materials omega-amino cyclohexane carboxy esters are produced.

### General Description for Synthesis of Omega-Amino Cyclohexylmethyl Carboxylate Derivatives (Scheme 7)

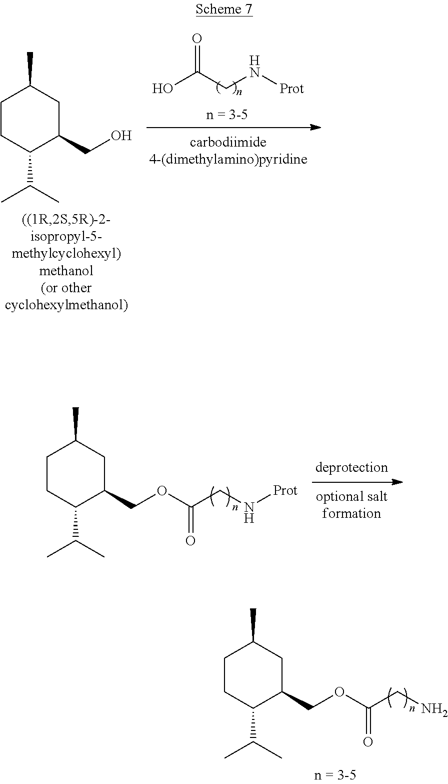

In general, omega-amino cyclohexylmethyl carboxylate analogs can be synthesized by the route described in Scheme 7. ((1R,2S,5R)-2-isopropyl-5-methylcyclohexyl)methanol or other cyclohexylmethanol can be obtained commercially or prepared by methods common in the art (E.g., Yang, Zhang, Ouyang; Xiangtan Daxue Ziran Kexue Xuebao (2009), 31(3), 96-100 and Kato, Ueda, Hashimoto; Agricultural and Biological Chemistry (1970), 34(1), 28-31). In a general description ((1R,2S,5R)-2-isopropyl-5-methylcyclohexyl)methanol or other cyclohexylmethanol can be esterified with N-protected ω-amino acids by methods common in the art (U.S. Pat. No. 8,637,547) to provide protected amino esters which can be further manipulated via deprotection or tested for TRPM8, TRPA1, and TRPV1 activity as independent chemical entities.

The protected amino esters can be deprotected to amino esters to provide omega-amino cyclohexylmethyl carboxylate esters.

### EXAMPLES

The following non-limiting EXAMPLES represent molecules synthesized using one or more methods of the present invention. All EXAMPLES were run at room temperature (RT), standard pressure and atmosphere, unless otherwise noted. The water used in the EXAMPLES was deionized water, unless otherwise noted.

### Example 1

### Ethyl (R)-3-(2-((tert-butoxycarbonyl)amino)acetamido)-3-phenylpropanoate

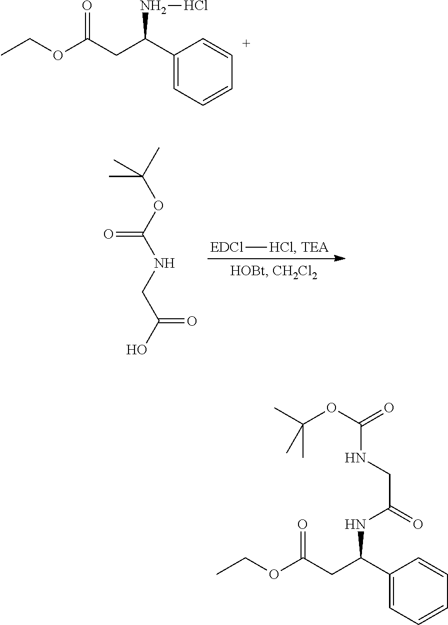

A dry 500 mL round bottom 3-neck flask equipped with a condenser having an outlet to a Firestone valve (positive nitrogen pressure), two stoppers, and a magnetic stir bar was charged with (R)-3-amino-3-phenylpropanoic acid ethyl ester hydrochloride (3.570 g, 15.5 mmol), Boc-Glycine (2.995 g, 17.10 mmol), 1 H-benzo [d] [1,2,3]-triazol-1-ol (HOBt) (2.310 g, 17.10 mmol), N-(3-dimethylaminopropyl)-N-ethyl-carbodiimide hydrochloride CAS 25952-53-8 (EDC-Hydrochloride) (3.575 g, 18.65 mmol) and 180 mL of anhydrous dichloromethane. After cycling between vacuum and nitrogen and leaving under a nitrogen atmosphere the mixture was stirred for 30 minutes, and then triethylamine (TEA) (4.72 g, 46.6 mmol) was added via syringe through a sidearm and stirring was continued overnight under nitrogen atmosphere. The reaction mixture was transferred to a separatory funnel and the flask was rinsed with 100 mL of ethyl acetate and 100 mL of water to make a complete transfer. An additional 400 mL of ethyl acetate and 400 mL of H₂O were added and mixed. The layers were separated and the aqueous layer was back extracted with 2×100 mL of ethyl acetate. The combined organic layers were washed with 2×500 mL of 1N HCl solution, 1× of 500 mL H₂O, 3×500 mL of saturated sodium bicarbonate solution, and 1×500 mL of saturated sodium chloride solution. The organic phase was dried with anhydrous magnesium sulfate, vacuum filtered and concentrated in vacuo to constant weight to yield 5.3 g as an oil: MS (ESI) m/z 351 (MH⁺).

### Example 2

### (R)-3-(2-((tert-butoxycarbonyl)amino)acetamido)-3-phenylpropanoic acid

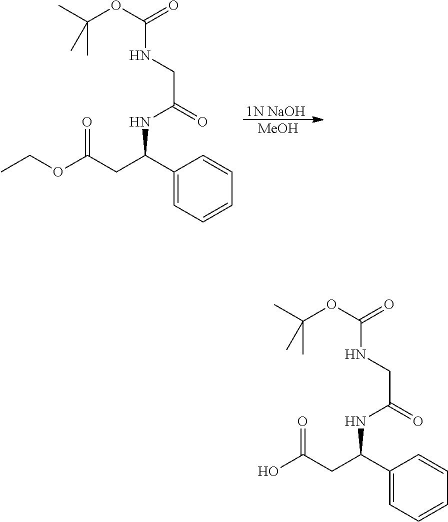

A 100 mL single neck round bottom flask was charge with ethyl (R)-3-(2-((tert-butoxycarbonyl)amino)acetamido)-3-phenylpropanoate (1.50 g, 4.28 mmol), 10 mL of methanol and 22 mL of 1N sodium hydroxide (22 mmol). The solution was stirred for 90 minutes then partially concentrated in vacuo to remove around 90% or more of the methanol. The resulting mixture was neutralized with 22 mL of 1 N HCl and extracted with 3×20 mL of methylene chloride. The combined organic layers were washed with 30 mL of saturated sodium chloride solution, dried over anhydrous sodium sulfate, filtered, and concentrated in vacuo at 40° C. and ˜5 mm Hg vacuum to yield the product as a white solid. MS (ESI) m/z 323 (MH⁺).

### Example 3

### (1S,2S,5R)-2-isopropyl-5-methylcyclohexyl (R)-3-(2-aminoacetamido)-3-phenylpropanoate hydrochloride

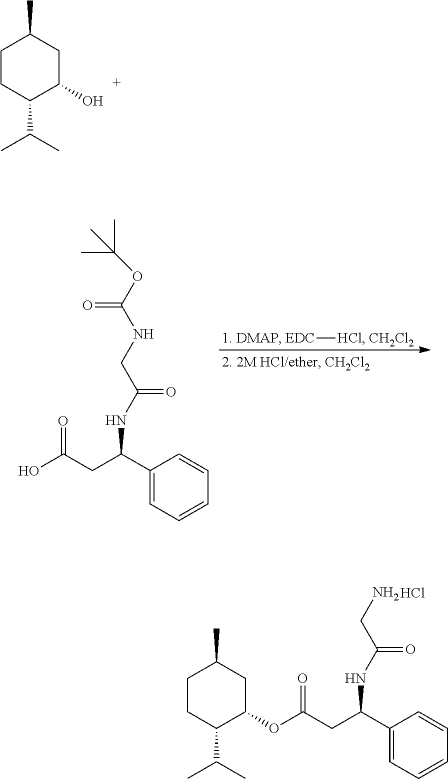

Step 1. A 25 mL round bottom 3-neck flask equipped with a condenser with an outlet to a Firestone valve (positive nitrogen pressure), two stoppers, and a magnetic stir bar was charged with (1S,2S,5R)-2-isopropyl-5-methylcyclohexan-1-ol CAS #2216-52-6 (0.0410 g, 0.260 mmol), (R)-3-(2-((tert-butoxycarbonyl)amino)acetamido)-3-phenylpropanoic acid (0.100 g, 0.310 mmol), N-(3-dimethylaminopropyl)-N-ethyl-carbodiimide hydrochloride (0.088 g, 0.459 mmol) and 2 mL of anhydrous dichloromethane. The solution was stirred under nitrogen for 5 minutes and 4-(dimethylamino)pyridine (DMAP) (0.0770 g, 0.630 mmol) was added in one portion through an open sidearm. The solution was stirred overnight under nitrogen. The reaction solution was transferred to a separatory funnel with 20 mL of dichloromethane and extracted with 2×20 mL of 1M HCl solution, 2×20 mL of saturated NaHCO₃, 1×20 mL of saturated sodium chloride adding 5 mL dichloromethane between washes. The organic layer was dried over anhydrous sodium sulfate, filtered, and concentrated in vacuo at 38° C. to constant weight to give (1S,2S,5R)-2-isopropyl-5-methylcyclohexyl (R)-3-(2-((tert-butoxycarbonyl)amino)acetamido)-3-phenylpropanoate as a white solid 0.1023 g: MS (ESI) m/z 461 (MH⁺).

Step 2. A dry 25 mL single neck round bottom flask containing a magnetic stir bar was charged with (1S,2S,5R)-2-isopropyl-5-methylcyclohexyl (R)-3-(2-((tert-butoxycarbonyl)amino)acetamido)-3-phenylpropanoate (0.0885 g, 0.192 mmol). Anhydrous dichloromethane (3 mL) was added and stirred under nitrogen atmosphere until the solid dissolved. After pouring in 2M hydrogen chloride in diethyl ether (8 mL, 16 mmol) the solution was stirred under nitrogen atmosphere at RT for 16 hours. The reaction mixture was concentrated in vacuo at 38° C. to constant weight. The product was recovered as a white solid 0.0762 g (100%): MS (ESI) m/z 361 (MH⁺ of free base).

### Example 4

### (1R,2R,5R)-2-isopropyl-5-methylcyclohexyl (R)-3-(2-aminoacetamido)-3-phenylpropanoate hydrochloride

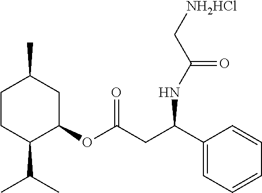

This compound was prepared, as described in EXAMPLE 3, using (1R,2R,5R)-2-isopropyl-5-methylcyclohexan-1-ol CAS #20752-34-5 in place of (1S,2S,5R)-2-isopropyl-5-methylcyclohexan-1-ol.

### Example 5

### (1S,2R,5S)-2-isopropyl-5-methylcyclohexyl (R)-3-(2-aminoacetamido)-3-phenylpropanoate hydrochloride

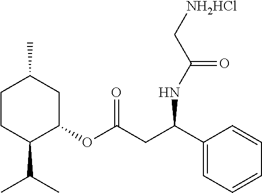

The above compound was prepared, as described in EXAMPLE 3, using (1S,2R,5S)-2-isopropyl-5-methylcyclohexan-1-ol CAS #15356-60-2 in place of (1S,2S,5R)-2-isopropyl-5-methylcyclohexan-1-ol.

### Example 6

### (1S,2R,5R)-2-isopropyl-5-methylcyclohexyl (R)-3-(2-aminoacetamido)-3-phenylpropanoate hydrochloride

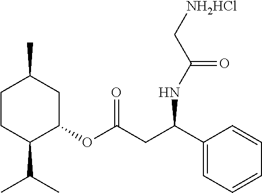

The above compound was prepared, as described in EXAMPLE 3, using (1S,2R,5R)-2-isopropyl-5-methylcyclohexan-1-01 CAS #23283-97-8 in place of (1S,2S,5R)-2-isopropyl-5-methylcyclohexan-1-ol.

### Example 7

### (1R,2S,5R)-2-isopropyl-5-methylcyclohexyl 6-((tert-butoxycarbonyl)amino)hexanoate

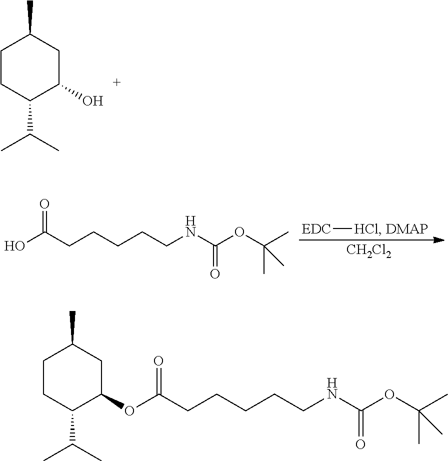

A 25 mL round bottom 3-neck round bottom flask equipped with a condenser with an outlet to a Firestone valve (positive nitrogen pressure) and a magnetic stir bar was charged with (1R,2S,5R)-2-isopropyl-5-methylcyclohexan-1-ol (0.1563 g, 1.0 mmol), 6-((tert-butoxycarbonyl)amino)hexanoic acid (0.2753 g, 1.19 mmol), N-(3-Dimethylaminopropyl)-N-ethyl-carbodiimide hydrochloride CAS 25952-53-8 (EDC-hydrochloride) (0.336 g, 1.75 mmol) and 5.2 mL of anhydrous dichloromethane. The mixture was stirred under nitrogen atmosphere for minutes until all reagents were in solution. DMAP (0.2933 g, 2.40 mmol) was added and stirring was continued overnight. The reaction was transferred to a separatory funnel and extracted with 2×25 mL of 1 M HCl, 2×25 mL of saturated NaHCO₃, and 1×20 mL of brine. The organic layer was dried over anhydrous sodium sulfate, vacuum filtered and concentrated on rotary evaporator (˜5 mmHg, 38° C.). The product was recovered as a colorless oil 0.3316 g. MS (ESI) m/z 370 (MH⁺).

### Example 8

### (1R,2S,5R)-2-isopropyl-5-methylcyclohexyl 6-aminohexanoate hydrochloride

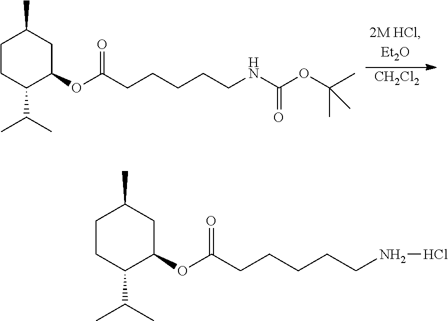

A 25 mL round bottom flask containing (1R,2S,5R)-2-isopropyl-5-methylcyclohexyl 6-((tert-butoxycarbonyl)amino)hexanoate (0.3116 g, 0.843 mmol) and 8 mL of anhydrous dichloromethane was stirred until all was in solution. A solution of 2.0 M hydrogen chloride in diethyl ether (15 mL, 30 mmol) was added and the solution was stirred under nitrogen atmosphere overnight. The mixture was concentrated on a rotary evaporator (38° C. and 5-7 mm Hg) followed by further drying under vacuum (0.1 mm Hg) to a constant weight. The product was recovered as a white solid 0.2475 g: MS (ESI) m/z 270 (MH⁺ of free base).

### Example 9

### (1R,2S,5R)-2-isopropyl-5-methylcyclohexyl (R)-3-((tert-butoxycarbonyl)amino)-3-phenylpropanoate

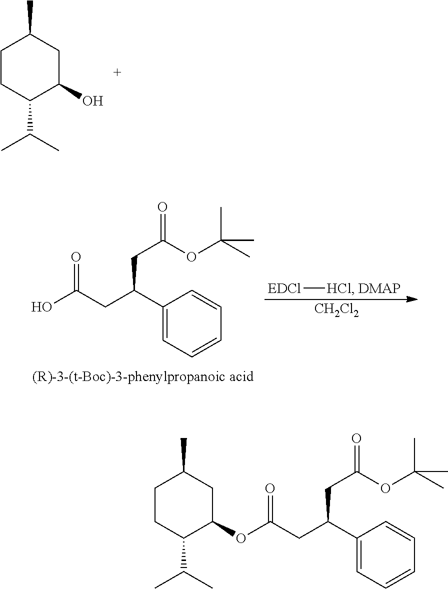

A dry 50 mL single neck round bottom flask equipped with a condenser with an outlet to a Firestone valve (positive nitrogen pressure) and a magnetic stir bar was charged with (1R,2S,5R)-2-isopropyl-5-methylcyclohexan-1-ol (0.492 g, 3.15 mmol), Boc-beta-Phe-OH CAS #161024-80-2 (0.995 g, 3.75 mmol), anhydrous dichloromethane (15 mL), 4-(dimethylamino)pyridine (DMAP) (0.917 g, 7.50 mmol) and N-(3-dimethylaminopropyl)-N-ethyl-carbodiimide hydrochloride CAS #25952-53-8 (EDC-hydrochloride) (0.8985 g, 4.69 mmol). The mixture was stirred under a nitrogen atmosphere overnight. The reaction was transferred to a separatory funnel and extracted with 2×80 mL of 1 M HCl, 2×80 mL of saturated NaHCO₃, and 1×65 mL of brine. The organic layer was dried over anhydrous sodium sulfate, vacuum filtered and concentrated on a rotary evaporator (˜5 mmHg, 38° C.). The product was recovered as a colorless oil 1.207 g: MS (ESI) m/z 404 (MH⁺).

### Example 10

### (1R,2S,5R)-2-isopropyl-5-methylcyclohexyl (R)-3-amino-3-phenylpropanoate hydrochloride

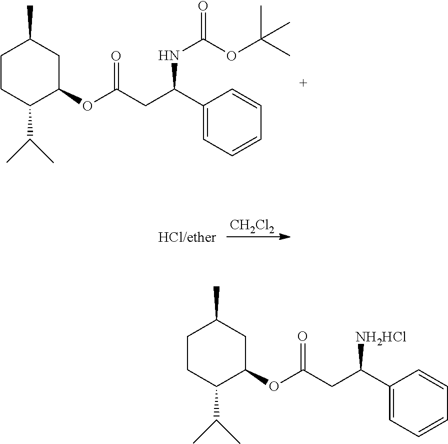

A 100 mL single neck round bottom flask equipped with a condenser with an outlet to a Firestone valve (positive nitrogen pressure) and a magnetic stir bar was charged with (1R,2S,5R)-2-isopropyl-5-methylcyclohexyl (R)-3-((tert-butoxycarbonyl)amino)-3-phenylpropanoate (1.207 g, 2.99 mmol) and 10 mL of anhydrous dichloromethane. The mixture was stirred under nitrogen atmosphere until the sample was in solution. A solution of 30 mL of 2M hydrogen chloride in diethyl ether (2.19 g, 60 mmol) was added, and the mixture was stirred under nitrogen overnight. The resulting mixture was concentrated in vacuo at 42° C. to constant weight to yield a white solid 1.001 g: MS (ESI) m/z 304 (MH⁺ of free base).

### Example 11

### (1R,2S,5R)-2-isopropyl-5-methylcyclohexyl (R)-3-(2-aminoacetamido)-3-phenylpropanoate hydrochloride

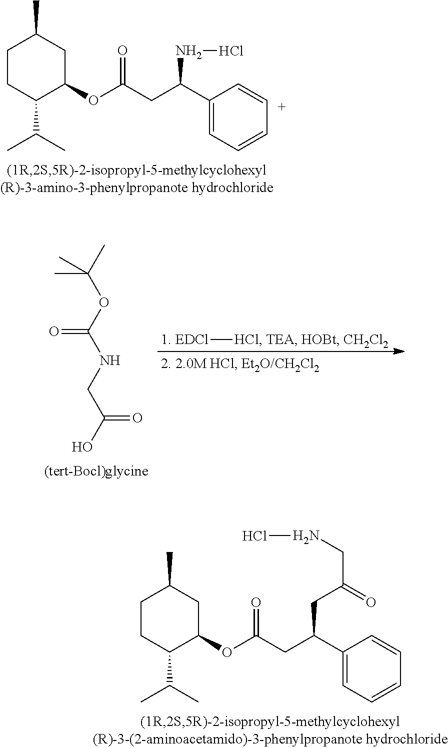

Step 1. Boc-Gly-OH (0.278 g, 1.59 mmol), 0.214 g of HOBt (1.59 mmol) and 0.332 g of EDC HCl (1.73 mmol) were placed in a 2-neck 100 mL RB flask fitted with a magnetic stir bar, Firestone valve (for argon and vacuum introduction), and a stopper. The flask was vacuum-argon cycled 5 times and left under positive argon pressure. A stock solution [A] containing 30 mL of CH₂Cl₂ and 0.6 mL of Et₃N (4.3 mmol) was prepared. After adding 10 mL of [A] through a side arm, with argon flowing through, the system was vacuum-argon cycled 5 times and stirred for 2 min at room temperature to dissolve the contents. After dissolving 0.490 g (1.44 mmol) of (1R,2S,5R)-2-isopropyl-5-methylcyclohexyl (R)-3-amino-3-phenylpropanoate hydrochloride in 10 mL of [A] it was added to the reaction flask through a side arm, with a stream of argon flowing through, and its container was rinsed with an additional 10 mL of [A] and added to the reaction flask. The light amber, homogeneous solution was stirred under argon for 3.25 h. the reaction contents were then transferred to a separatory funnel. The CH₂Cl₂ solution was extracted with 1N HCl (10 mL+2 mL of methanol to break up the emulsion), another 10 mL of 1N HCl (organic layer was cloudy), 10 mL of H2O (organic layer was still cloudy), saturated NaHCO₃ solution (2×10 mL, both layers were clear), and 15 mL of a saturated NaCl solution. The organic layer was dried over Na₂SO₄ overnight. The Na₂SO₄ was removed by filtration and solvent was removed via a rotary evaporator under reduced pressure (5-10 mm Hg) at 40° C. to give 0.615 g of (1R,2S,5R)-2-isopropyl-5-methylcyclohexyl (R)-3-(2-((tert-butoxycarbonyl)amino)acetamido)-3-phenylpropanoate as an off-white solid. MS (ESI) m/z 461 (MH⁺, 100%), 405 (MH⁺−56, 50%). This material was then used in Step 2.

Step 2. A 50 mL round bottom flask with a magnetic stir bar was charged with 15 mL of anhydrous CH₂Cl₂ and 0.365 g (0.792 mmol) of (1R,2S,5R)-2-isopropyl-5-methylcyclohexyl (R)-3-(2-((tert-butoxycarbonyl)amino)acetamido)-3-phenylpropanoate. A solution of 2.0 M HCl in diethyl ether (10 mL, 20 mmol) was added while stirring, then the flask was capped. The reaction solution became cloudy white and was stirred an additional 23 hours before the solvent was removed via a rotary evaporator under reduced pressure (5-10 mm Hg, up to 40° C.) to give 0.315 g of (1R,2S,5R)-2-isopropyl-5-methylcyclohexyl (R)-3-(2-aminoacetamido)-3-phenylpropanoate hydrochloride as a white solid: MS (ESI) m/z 361 (MH⁺ of free base).

### Example 12

### (1R,2S,5R)-2-isopropyl-5-methylcyclohexyl (R)-3-((S)-2-((tert-butoxycarbonyl)amino)propanamido)-3-phenylpropanoate

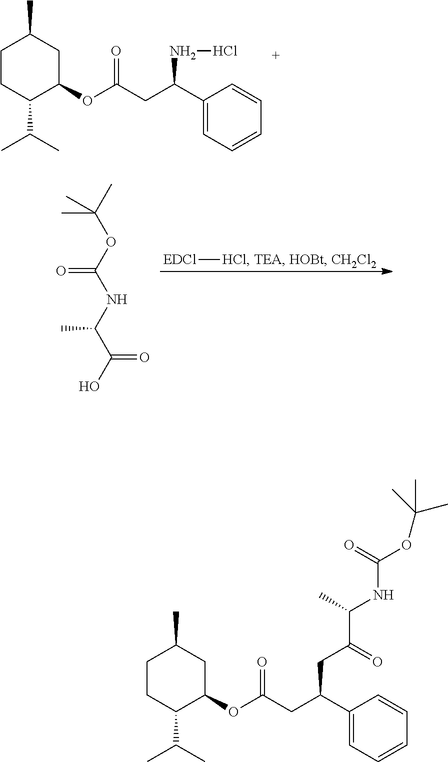

A dry 50 mL 3-neck round bottom flask equipped with a condenser with an outlet to a Firestone valve (positive nitrogen pressure) and a magnetic stir bar was charged with (1R,2S,5R)-2-isopropyl-5-methylcyclohexyl (R)-3-amino-3-phenylpropanoate hydrochloride (0.1001 g, 0.295 mmol), Boc-L-alanine (0.0585 g, 0.309 mmol), HOBt (0.042 g, 0.309 mmol), N-(3-dimethylaminopropyl)-N-ethyl-carbodiimide hydrochloride CAS 25952-53-8 (EDC-hydrochloride) (0.059 g, 0.309 mmol), anhydrous dichloromethane (10 mL) and triethylamine (0.032 g, 0.318 mmol). The heterogeneous mixture was stirred under a nitrogen atmosphere overnight. The reaction mixture was transferred to a separatory funnel containing ethyl acetate (60 mL) and distilled water (60 mL). The aqueous layer was separated and extracted with 2×20 mL ethyl acetate. The combined organic layers were washed with 1N HCl solution (2×50 mL), distilled water (1×50 mL), saturated sodium bicarbonate solution (3×50 mL), and saturated sodium chloride solution (1×50 mL). The organic solution was dried over anhydrous sodium sulfate, filtered and concentrated under vacuum at 40° C. to give a white solid 0.1239 g: MS (ESI) m/z 475 (MH⁺).

### Example 13

### (1R,2S,5R)-2-isopropyl-5-methylcyclohexyl (R)-3-((R)-2-((tert-butoxycarbonyl)amino)propanamido)-3-phenylpropanoate

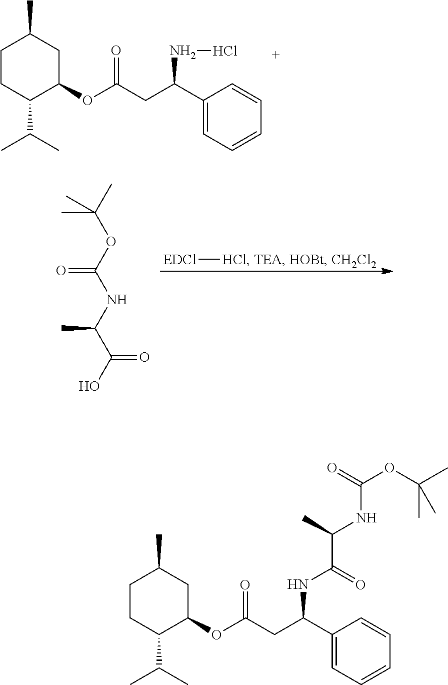

This compound was prepared following the same procedure as described for EXAMPLE 12, except Boc-D-Alanine was used in place of Boc-L-Alanine.

### Example 14

### (1R,2S,5R)-2-isopropyl-5-methylcyclohexyl (R)-3-((S)-2-aminopropanamido)-3-phenylpropanoate hydrochloride

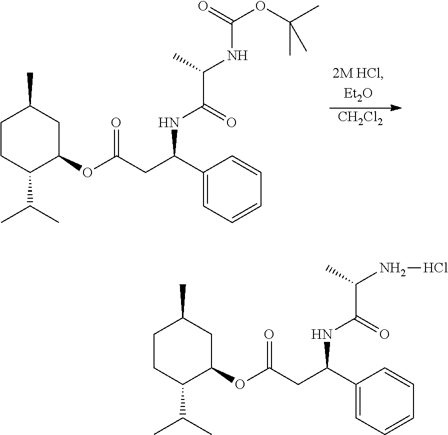 [SMILES: CC(C)[C@@H]1CC[C@@H](C)C[C@H]1OC(=O)C[C@@H](NC(=O)[C@H](C)NC(=O)OC(C)(C)C)c1ccccc1 ; "CH2Cl2" ; CC(C)[C@@H]1CC[C@@H](C)C[C@H]1OC(=O)C[C@@H](NC(=O)[C@H](C)NCl)c1ccccc1]

Anhydrous dichloromethane (4 mL) was added to a dry 25 mL single neck round bottom flask containing (1R,2S,5R)-2-isopropyl-5-methylcyclohexyl (R)-3-((S)-2-((tert-butoxycarbonyl)amino)propanamido)-3-phenylpropanoate (0.1239 g, 0.261 mmol) and the mixture was stirred under a nitrogen atmosphere. A solution of 2M hydrogen chloride in diethyl ether (20 mL, 40 mmol) was added to the flask and stirred overnight at ambient conditions. The solution was concentrated in vacuo to constant weight. The product was recovered as a white powder 0.0987 g: MS (ESI) m/z 375 (MH⁺ of the free base).

### Example 15

### (1R,2S,5R)-2-isopropyl-5-methylcyclohexyl (R)-3-((R)-2-aminopropanamido)-3-phenylpropanoate hydrochloride

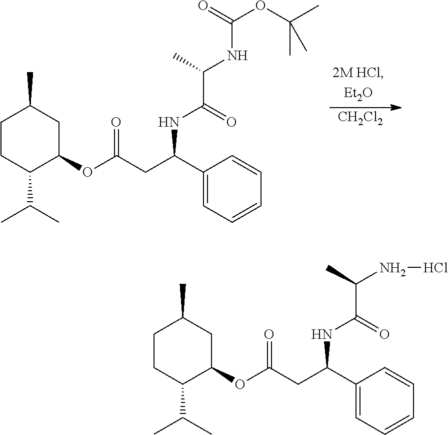 [SMILES: CC(C)[C@@H]1CC[C@@H](C)C[C@H]1OC(=O)C[C@@H](NC(=O)[C@H](C)NC(=O)OC(C)(C)C)c1ccccc1 ; "CH2Cl2" ; CC(C)[C@@H]1CC[C@@H](C)C[C@H]1OC(=O)C[C@@H](NC(=O)[C@@H](C)NCl)c1ccccc1]

This compound was prepared following the same procedure as described for EXAMPLE 14, except (1R,2S,5R)-2-isopropyl-5-methylcyclohexyl (R)-3-((R)-2-((tert-butoxycarbonyl)amino)propanamido)-3-phenylpropanoate was used in place of (1R,2S,5R)-2-isopropyl-5-methylcyclohexyl (R)-3-((S)-2-((tert-butoxycarbonyl)amino)propanamido)-3-phenylpropanoate.

### Example 16

### (Racemic)-trans-2-isopropyl-cis-5-methylcyclohexyl 2-(2-aminoacetamido)benzoate hydrochloride

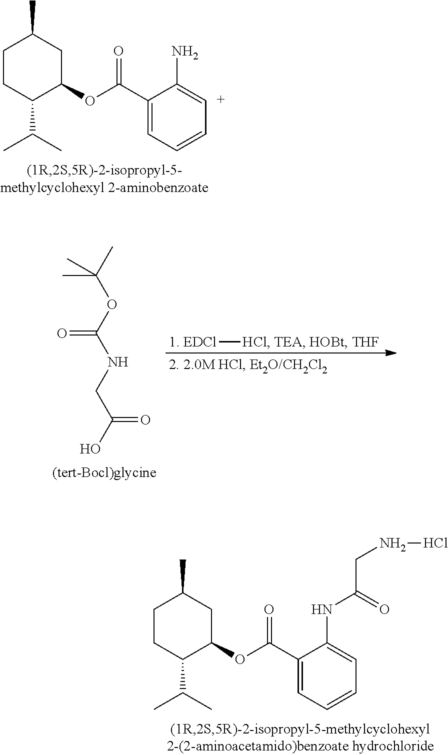

Step 1. Boc-Gly-OH (0.771 g, 4.40 mmol), 0.594 g of HOBt (4.40 mmol) and 0.844 g of EDC HCl (4.40 mmol) were placed in a 3-neck 250 mL RB flask fitted with a magnetic stir bar, a condenser topped with a Firestone valve (for argon and vacuum introduction), and two stoppers. After adding 25 mL of tetrahydrofuran (THF) the flask was vacuum-argon cycled 5 times and left under positive argon pressure. Trimethylamine (0.62 mL, 4.40 mmol) was added through a side arm with argon flowing through followed by the addition of 1.102 g (4.00 mmol) of (racemic)-trans-2-isopropyl-cis-5-methylcyclohexyl 2-aminobenzoate in 55 mL of THF. The system was vacuum-argon cycled five times to give a cloudy white suspension at room temperature stirring under an argon atmosphere overnight. After seeing only a trace of product by TLC 0.395 g of 4-(dimethylamino)pyridine (DMAP, 3.23 mmol) was added, the flask was fitted with a heating mantle, and heated at 65° C. under argon for 10 days. After cooling to room temperature the sticky solids and solvent in the reaction flask were dissolved in 50 mL of 1 M HCl and added to a separatory funnel containing 75 mL of ethyl ether. The ether layer was successively extracted with another 30 mL of 1 M HCl, 50 mL of water (plus 10 mL of saturated NaCl to speed phase separation), 2×30 mL of saturated NaHCO₃, and 30 mL of saturated NaCl solution. The ether solution was dried over. The Na₂SO₄ was removed by filtration and solvent was removed via a rotary evaporator under reduced pressure (5-10 mm Hg) at 40° C. to give 1.126 g of thick, colorless residue. This residue was purified by flash chromatography on 20.2 g of Merck 60 silica gel (16×187 mm column) eluting with 3:1 hexane: ethyl acetate to provide 0.110 g of (racemic)-trans-2-isopropyl-cis-5-methylcyclohexyl 2-(2-((tert-butoxycarbonyl)amino)acetamido)benzoate as a colorless oil. MS (ESI) m/z 433 (MH⁺, 100%), 377 (MH⁺−56, 10%). This material was used in Step 2.

Step 2. A 50 mL pear-shaped flask was charged with 0.110 g of (racemic)-trans-2-isopropyl-cis-5-methylcyclohexyl 2-(2-((tert-butoxycarbonyl)amino)acetamido)benzoate in 4 mL of chloroform along with a magnetic stir bar. The addition of 3 mL of 2 M HCl in ether was followed by stirring for 18 h. The solvent was removed via a rotary evaporator under reduced pressure (5-10 mm Hg) at 40° C. to give 0.088 g of (racemic)-trans-2-isopropyl-cis-5-methylcyclohexyl 2-(2-aminoacetamido)benzoate hydrochloride white solid: MS (ESI) m/z 433 (MH⁺ of free base).

### Example 17

### (1R,2S,5R)-2-isopropyl-5-methylcyclohexyl glycylphenylalaninate hydrochloride

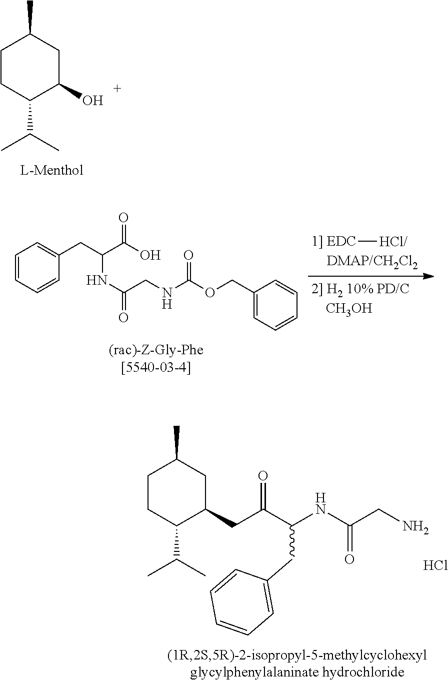

Step 1. The following reagents were weighed out together in a 50 mL RB flask with magnetic stir bar: 0.156 g (1.00 mmol) of L-menthol, 0.534 g (1.50 mmol) of (rac)-Z-Gly-Phe [5540-03-4], 0.594 g (3.10 mmol) of EDC HCl, and 0.752 g (6.16 mmol) of 4-(dimethylamino)pyridine

(DMAP). After the addition of 10 mL of methylene chloride the flask was capped and the homogeneous mixture was stirred for 23 h. Analysis by thin layer chromatography (SiO₂, Et₂O) showed remaining menthol. An additional 0.238 g (1.24 mmol) of EDC HCl was added and stirring was continued for 3 days. The reaction was poured into 20 mL of 1 M HCl plus 25 mL Et₂O and the phases were separated. The organic layer was extracted with another 20 mL of 1 M HCl, 2×20 mL of half-saturated NaHCO₃, 10 mL water, and 20 mL of saturated NaCl solution. After drying over Na₂SO₄ the crude product was purified by flash chromatography (16 g of silica gel 60, 14.5 mm×7″ column). Elution began with 1:1 Et₂O:pentane (100 mL) followed by 160 mL of 2:1 Et₂O:pentane to obtain 0.295 g of (1R,2S,5R)-2-isopropyl-5-methylcyclohexyl ((benzyloxy)carbonyl)glycylphenylalaninate, as a white semi-solid: MS (ESI) m/z 495 (MH⁺).

Step 2. A 7 mL stainless steel Parr pressure reactor was charged with 0.0935 g (0.188 mmol) of (1R,2S,5R)-2-isopropyl-5-methylcyclohexyl ((benzyloxy)carbonyl)glycylphenylalaninate, 0.017 g of 10% palladium on carbon, 4 mL of methanol, and a small magnetic stir bar. The reactor was flushed with argon before pressurizing with 100-150 psi of hydrogen and venting five times. The system was then pressurized with 120 psi of hydrogen and stirred for 29 h. The system was then vented, flushed with argon (vac/Ar cycled with Firestone valve connected to vent), filtered through 0.45μ Nylon centrifuge filters (3000 rpm), and the filters were rinsed with 6 mL of methanol. The methanol was removed via a rotary evaporator under reduced pressure (5-10 mm Hg) at 40° C. to give 0.057 g of the product as the free base: MS (ESI) m/z 361 (MH⁺, 40%), m/z 223 (100%). The free base was converted to the final hydrochloride product, (1R,2S,5R)-2-isopropyl-5-methylcyclohexyl glycylphenylalaninate hydrochloride, by taking the free base up in 5 mL of methylene chloride, adding 3 mL of 2M HCl in Et₂O, agitating for 5 min, and concentrating via a rotary evaporator under reduced pressure (5-10 mm Hg) at 40° C.

### Example 18

### (1R,2S,5R)-2-isopropyl-5-methylcyclohexyl 3-((2-aminoethyl)amino)-3-phenylpropanoate dihydrochloride

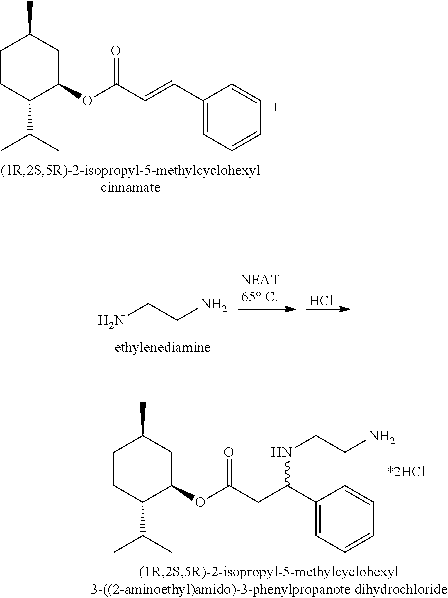

Ethylenediamine (0.315 g, 5.24 mmol) and 0.488 g (1.70 mmol) of (1R,2S,5R)-2-isopropyl-5-methylcyclohexyl cinnamate (see Angewandte Chemie, International Edition, 55(1), 218-222; 2016; Journal of the American Chemical Society, 129(47), 14775-14779; 2007) were weighed out together in 5 mL pear-shaped flask to give a homogeneous mixture. The flask was connected to a Firestone valve (for nitrogen and vacuum introduction) and after cycling five times between vacuum and nitrogen the system was left under a positive nitrogen atmosphere, and the flask was placed in a 65-66° C. oil bath (without stirring) for 4 days. Upon cooling the reaction mixture was combined with 5 mL of water and 15 mL of Et₂O in a separatory funnel. The phases were separated, the organic phase was extracted with 2×10 mL more water, 10 mL of saturated NaCl solution and the Et₂O solution was dried over Na₂SO₄. The Na₂SO₄ was removed by filtration and solvent was removed via a rotary evaporator under reduced pressure (5-10 mm Hg) to give 0.487 g of oil which was purified by flash chromatography on 13.7 g of silica gel 60 (6″×14.5 mm column) eluting with 90:10:1 chloroform (containing 1% ethanol):methanol:ammonium hydroxide (29% aqueous) to provide 0.268 g of the free base as a soft solid. MS (ESI) m/z 347 (MH⁺, 75%), m/z 209 (100%). The free base was taken up in 10 mL of methylene chloride, 4 mL of 2.0 M HCl in Et₂O was added, the mixture was capped and agitated for 25 min, and then concentrated via a rotary evaporator under reduced pressure (5-10 mm Hg) at 40° C. to give the salt form, (1R,2S,5R)-2-isopropyl-5-methylcyclohexyl 3-((2-aminoethyl)amino)-3-phenylpropanoate dihydrochloride as a white solid.

### Example 19

### (1R,2S,5R)-2-isopropyl-5-methylcyclohexyl 3-phenyl-3-((pyridin-2-ylmethyl)amino)propanoate dihydrochloride

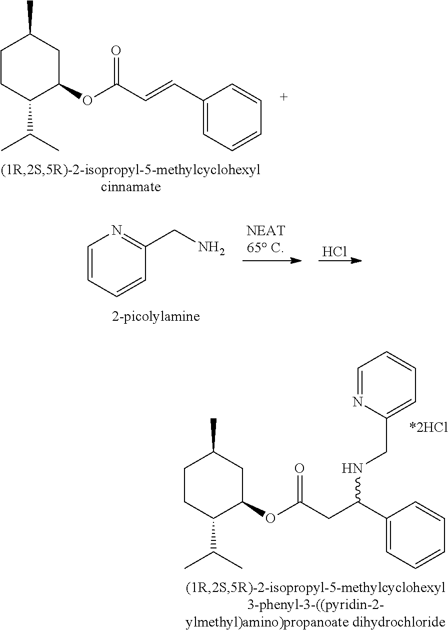 [SMILES: CC(C)[C@@H]1CC[C@@H](C)C[C@H]1OC(=O)/C=C/c1ccccc1 ; NCc1ccccn1 ; Cl ; CC(C)[C@@H]1CC[C@@H](C)C[C@H]1OC(=O)CC(NCc1ccccn1)c1ccccc1]

2-Picolylamine (0.607 g, 5.61 mmol) and 0.511 g (1.78 mmol) of (1R,2S,5R)-2-isopropyl-5-methylcyclohexyl cinnamate (see Angewandte Chemie, International Edition, 55(1), 218-222; 2016; Journal of the American Chemical Society, 129(47), 14775-14779; 2007) were weighed out together in 5 mL pear-shaped flask to give a homogeneous mixture. The flask was connected to a Firestone valve (for nitrogen and vacuum introduction) and after cycling five times between vacuum and nitrogen the system was left under a positive nitrogen atmosphere, and the flask was placed in a 65-66° C. oil bath (without stirring) for 4 days. Upon cooling the reaction mixture was combined with 10 mL of water and 15 mL of Et₂O. The phases were separated, the organic phase was extracted with 2×10 mL more water, 10 mL of saturated NaCl solution and the Et₂O solution was dried over Na₂SO₄. The Na₂SO₄ was removed by filtration and solvent was removed via a rotary evaporator under reduced pressure (5-10 mm Hg) to give 0.374 g of a thick oil which was purified by flash chromatography on 13.7 g of silica gel 60 (6″×14.5 mm column) eluting with 1:1 hexanes:ethyl acetate to provide 0.067 g of the free base. MS (ESI) m/z 395 (MH⁺). The free base was taken up in 8 mL of methylene chloride, 2 mL of 2.0 M HCl in Et₂O was added, the mixture was capped and agitated for 25 min, and then concentrated via a rotary evaporator under reduced pressure (5-10 mm Hg) at 40° C. to give the salt form, (1R,2S,5R)-2-isopropyl-5-methylcyclohexyl 3-phenyl-3-((pyridin-2-ylmethyl)amino)propanoate dihydrochloride as a white solid.

### Example 20

### (1R,2S,5R)-2-Isopropyl-5-methylcyclohexane-1-carbonyl chloride

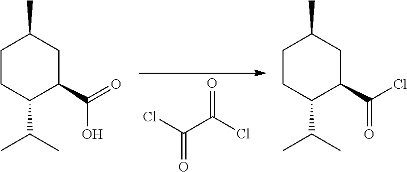

A 250 mL single neck round bottom flask was charged with (1R,2S,5R)-2-Isopropyl-5-methylcyclohexanecarboxylic acid (10.001 gram, 0.054 mol), and 92 mL of oxalyl chloride (138 g, 1.08 mol). The solution was stirred under a positive pressure nitrogen atmosphere for 18 hours then concentrated under vacuum. The product was recovered as 10.42 grams of a clear, colorless liquid.

### Example 21

### (S)-2-((R)-2-((tert-butoxycarbonyl)amino)propanamido)-2-phenylethyl (1R,2S,5R)-2-isopropyl-5-methylcyclohexane-1-carboxylate

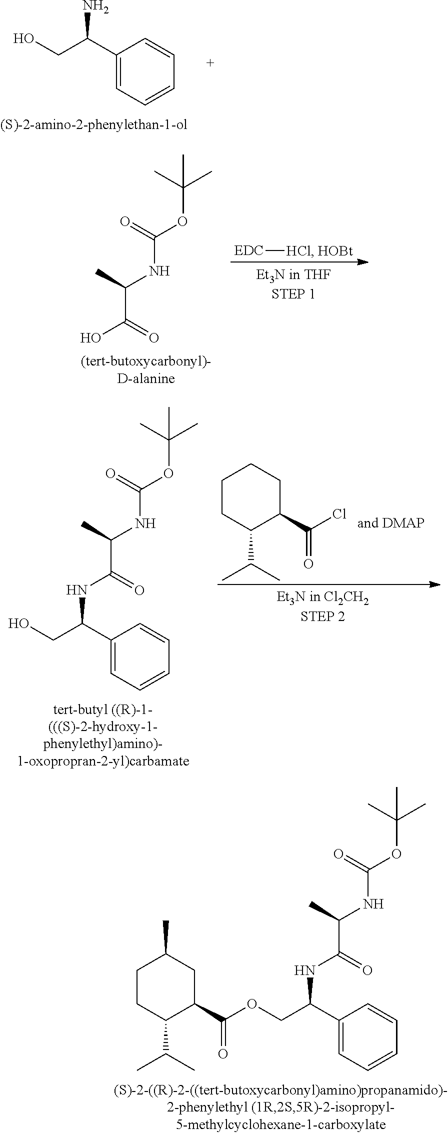

Step 1—A 500 mL round-bottom flask (A) containing a magnetic stir bar and 50 mL of tetrahydrofuran (THF) was charged with N-(3-dimethylaminopropyl)-N′-ethylcarbodiimide hydrochloride (5.15 g EDC-HCl, 27 mmol), triethylamine (3.05 g, 30 mmol) dissolved in an additional 20 mL of THF, 1H-benzo[d][1,2,3] triazol-1-ol (HOBt, 3.62 g, 27 mmol) dissolved in an additional 30 mL of THF, and (tert-butoxycarbonyl)-D-alanine (BOC-D-Ala-OH, 4.56 g, 24 mmol) dissolved in an additional 20 mL of THF. A second 500 mL round-bottom flask (B) was charged with (S)-2-amino-2-phenylethan-1-ol (3.09 g, 22 mmol) and 30 mL of THF. The contents of flask A were transferred into flask B by pipet. The reaction vessel was connected to an oil bubbler and the heterogeneous mixture was stirred under static nitrogen atmosphere at 20-25° C. for 24 hours. The heterogeneous reaction mixture was transferred to a 2 liter separatory funnel containing 100 mL of ethyl acetate (EtOAc) and 100 mL of water, and the reaction products were observed to dissolve completely. Following the initial extraction the aqueous layer was isolated and extracted with 2×50 mL of EtOAc. The combined organic layers were subsequently extracted with 1 N HCl (3×100 mL), saturated sodium bicarbonate (3×100 mL), and saturated potassium chloride (100 mL). The recovered organic layer was dried over anhydrous sodium sulfate overnight. After filtration the solvent was subsequently removed in vacuo (40-45° C.) to give 6.2 g of a white solid, tert-butyl ((R)-1-(((S)-2-hydroxy-1-phenylethyl)amino)-1-oxopropan-2-yl)carbamate: MS (ESI) m/z 309 (MH⁺).

Step 2—A vial (A) was charged with 4-(dimethylamino)pyridine (DMAP, 0.072 g, 0.5 mmol), 5 mL of methylene chloride and triethylamine (790 μL). A 100 mL round-bottom flask (B) was charged with (1R,2S,5R)-2-isopropyl-5-methylcyclohexane-1-carbonyl chloride (1.12 g, 5.5 mmol) and 22 mL of CH₂Cl₂. A 250 mL, three-neck round-bottom flask (C) was charged with tert-butyl ((R)-1-(((S)-2-hydroxy-1-phenylethyl) amino)-1-oxopropan-2-yl)carbamate (1.50 g, 4.9 mmol) and 5 mL CH₂Cl₂ and a magnetic stir bar. The resulting solution in vial A was added to the reactor flask C by pipet. The reactor was purged with dry nitrogen and immersed in an ice bath. The resulting solution in flask B was added to the reactor (C) via transfer cannula and dry nitrogen over 5 minutes. The flask B was rinsed with 20 mL of CH₂Cl₂ and the rinse was added to the reactor flask C via transfer cannula and dry nitrogen. The reaction vessel was connected to an oil bubbler and stirred under a static nitrogen atmosphere at 20-25° C. for 22 hours. The reactor contents were transferred to a 250 mL separatory funnel. The organic phase was extracted with 0.1 M HCl (3×50 mL), saturated sodium bicarbonate (2×50 mL), 1.0 M sodium hydroxide (50 mL) and saturated potassium chloride (2×50 mL). The recovered organic layer was dried over anhydrous sodium sulfate. After filtration the solvent was subsequently removed in vacuo (40-45° C.) to give 1.0 g of (S)-2-((R)-2-((tert-butoxycarbonyl)amino)propanamido)-2-phenylethyl (1R,2S,5R)-2-isopropyl-5-methylcyclohexane-1-carboxylate as a faint yellow colored and viscous liquid. MS (ESI) m/z 475 (MH⁺).

Using the procedure described above (Steps 1 and 2) and the appropriate Boc protected amino acid the following protected menthyl-carboxylate-ester was also prepared:
(S)-2-((S)-2-((tert-butoxycarbonyl)amino)propanamido)-2-phenylethyl (1R,2S,5R)-2-isopropyl-5-methylcyclohexane-1-carboxylate: MS (ESI) m/z=475 (MH⁺).

### Example 22

### (S)-2-((R)-2-aminopropanamido)-2-phenylethyl (1R,2S,5R)-2-isopropyl-5-methylcyclohexane-1-carboxylate hydrochloride

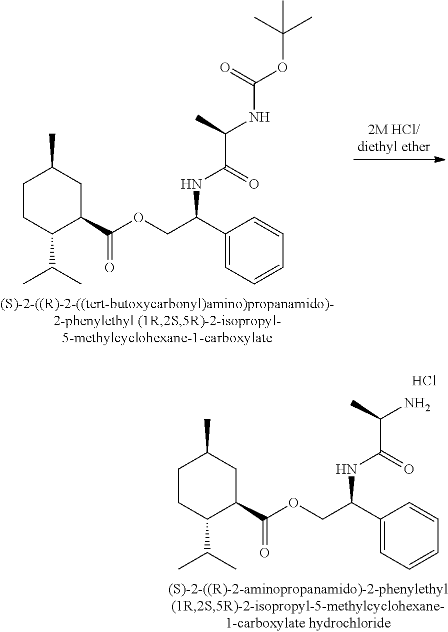 [SMILES: CC(C)[C@@H]1CC[C@@H](C)C[C@H]1C(=O)OC[C@@H](NC(=O)[C@@H](C)NC(=O)OC(C)(C)C)c1ccccc1 ; Cl ; CC(C)[C@@H]1CC[C@@H](C)C[C@H]1C(=O)OC[C@@H](NC(=O)[C@@H](C)N)c1ccccc1]

A 250 mL round-bottom flask containing a magnetic stir bar was charged with (S)-2-((R)-2-((tert-butoxycarbonyl)amino)propanamido)-2-phenylethyl (1R,2S,5R)-2-isopropyl-5-methylcyclohexane-1-carboxylate (0.25 g, 0.53 mmol) and 2 mL of 2.0 M HCl in diethyl ether (4 mmol). The reaction vessel was connected to an oil bubbler, and the reactor contents were stirred under a static nitrogen atmosphere and at 20-25° C. for 20 hours. The reaction progress was monitored using TLC, where the solvent, 1:1 hexane and ethyl acetate, resolved the starting protected amide at R_{f}˜0.6 from the hydrochloride ester product at R_{f}=0. Following the reaction time the diethyl ether solvent and any volatile reaction by-products were removed in vacuo (40-45° C.) to give 0.196 g of the title compound as an off-white solid: the MS (ESI) m/z 375 (MH⁺).

Using the procedure described above and the appropriate Boc protected amino acid, the following menthyl-carboxylate-ester was prepared:
(S)-2-((S)-2-aminopropanamido)-2-phenylethyl (1R,2S,5R)-2-isopropyl-5-methylcyclohexane-1-carboxylate hydrochloride: MS (ESI) m/z=375 (MH⁺ of the free base).

### Example 23

### (S)-2-(2-((tert-butoxycarbonyl)amino)acetamido)-2-phenylethyl (1R,2S,5R)-2-isopropyl-5-methylcyclohexane-1-carboxylate

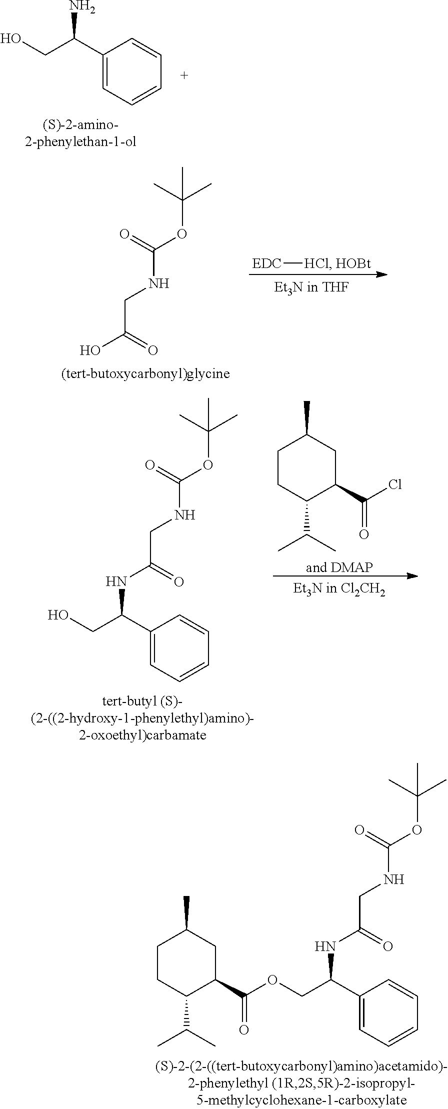

Step 1—A 500 mL round-bottom flask (A) was charged with a magnetic stir bar, 30 mL of tetrahydrofuran (THF), N-(3-dimethylaminopropyl)-N′-ethylcarbodiimide hydrochloride (3.27 grams EDC-HCl, 17 mmol), triethylamine (1.90 g Et₃N, 19 mmol) dissolved in an additional 13 mL of THF, 1H-benzo [d][1,2,3] triazol-1-ol (2.36 g HOBt, 17 mmol) dissolved in an additional 20 mL of THF and tert-butoxycarbonyl)glycine (2.78 g BOC-Gly-OH, 16 mmol) dissolved in an additional 12 mL of THF. A separate, 100 mL round-bottom flask (B) was charged with (S)-2-amino-2-phenylethan-1-ol (1.96 g, 14 mmol) and 20 mL of THF. An additional 3.0 g of triethylamine (Et₃N, 30 mmol) was added to flask A. The contents of flask B were transferred into flask A by pipet over a time period of 2 minutes. The reaction vessel was stoppered with a septum and the heterogeneous mixture was mixed at 500 r.p.m. and 20-25° C. for 22 hours. The heterogeneous reaction mixture was transferred to a 2 liter separatory funnel following the reaction period. The separatory funnel contained 100 mL of ethyl acetate (EtOAc) and 100 mL of water. The reaction products were observed to dissolve completely upon addition to the funnel. The water phase was extracted with 2×50 mL of EtOAc. The combined organic phases were subsequently extracted with 1 N HCl (3×100 mL), saturated sodium bicarbonate (1×100 mL), and saturated potassium chloride (100 mL). The recovered organic layer was dried over anhydrous sodium sulfate. The organic phase was filtered through Whatman #4 filter to remove the drying agent. The solvent was removed in vacuo (40-45° C.) to give 3.6 g of a sticky residue which was subsequently dissolved in methanol and vacuum stripped two additional times to give 3.4 g of a light amber colored and sticky residue, tert-butyl (S)-(2-((2-hydroxy-1-phenylethyl)amino)-2-oxoethyl)carbamate: MS (ESI) m/z 295 (MH⁺).

Step 2—A 250 ml round-bottom flask (A) was charged with a stir bar, tert-butyl (S)-(2-((2-hydroxy-1-phenylethyl)amino)-2-oxoethyl)carbamate (1.94 g, 6.6 mmol), 4-(dimethylamino)pyridine (0.102 g DMAP, 0.8 mmol) dissolved in 5 mL of methylene chloride and triethylamine (0.94 g TEA, 9.3 mmol). The headspace of Flask A was purged with dry nitrogen and the flask was immersed in an ice bath with magnetic mixing set to 200 r.p.m. A separate 100 mL round-bottom flask (B) was charged with (1R,2S,5R)-2-isopropyl-5-methylcyclohexane-1-carbonyl chloride (1.61 g L-Men-AC, 7.9 mmol) and 25 mL of CH₂Cl₂. The resulting solution in flask B was added to the reactor flask A by pipet over five minutes while purging the reactor headspace with dry nitrogen. Flask B was rinsed with 20 mL of methylene chloride and this was added to flask A. The reaction vessel was connected to an oil bubbler and stirred at 200 r.p.m. under a static nitrogen atmosphere and melting ice bath/20-25° C. for 24 hours. The reactor contents were transferred to a 250 mL separatory funnel following the reaction period. The organic phase was extracted with 1.0 M HCl (3×50 mL), 1.0 N NaOH (3×50 mL) and saturated potassium chloride (1×50 mL). The recovered organic layer was dried over anhydrous sodium sulfate. The organic phase was filtered through Whatman #4 filter to remove the drying agent. The solvent was removed in vacuo (30-35° C.) to give 2.5 g of crude product. The crude product was purified by flash chromatography in which the stationary phase was a 6 inch column×25 mm diameter of Silica Gel 60 and the mobile phase was 70:30 v/v hexanes and ethyl acetate. The mixed solvent was able to resolve the target compound, (S)-2-(2-((tert-butoxycarbonyl)amino)acetamido)-2-phenylethyl (1R,2S,5R)-2-isopropyl-5-methylcyclohexane-1-carboxylate, an off-white solid, from the byproducts: MS (ESI) m/z 461 (MH⁺).

### Example 24

### (S)-2-(2-aminoacetamido)-2-phenylethyl (1R,2S,5R)-2-isopropyl-5-methylcyclohexane-1-carboxylate hydrochloride

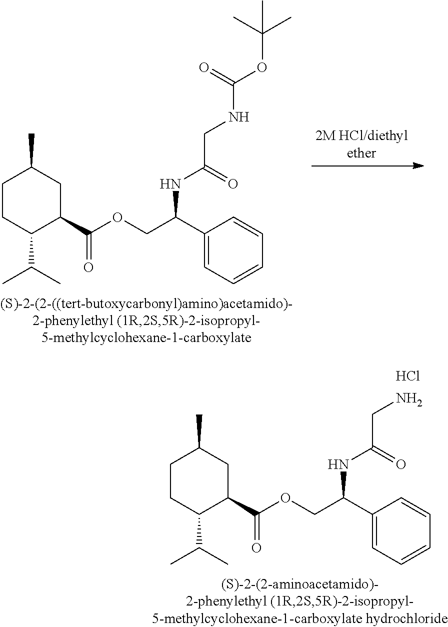 [SMILES: CC(C)[C@@H]1CC[C@@H](C)C[C@H]1C(=O)OC[C@@H](NC(=O)CNC(=O)OC(C)(C)C)c1ccccc1 ; Cl ; CC(C)[C@@H]1CC[C@@H](C)C[C@H]1C(=O)OC[C@@H](NC(=O)CN)c1ccccc1]

A 250 mL round-bottom flask was charged with a magnetic stir bar, (S)-2-(2-((tert-butoxycarbonyl)amino)acetamido)-2-phenylethyl (1R,2S,5R)-2-isopropyl-5-methylcyclohexane-1-carboxylate (1.0 g, 2.2 mmol) and 15 mL of 2.0 M HCl dissolved in diethyl ether (30 mmol HCl). The reaction vessel was connected to an oil bubbler and the reactor contents were stirred at 200 r.p.m. under a static nitrogen atmosphere and 20-25° C. An additional 10 mL of 2.0 M HCl dissolved in diethyl ether (20 mmol HCl) was added to the flask following a reaction period of approximately five hours. The reaction was allowed to continue to mix for a total of 24 hours. The diethyl ether solvent and any volatile reaction by-products were removed in vacuo (30-35° C.) to give 0.86 g of the title compound as an off-white solid: the MS (ESI) m/z was 361 (MH⁺ of the free base).

### Example 25

### (R)-2-(2-((tert-butoxycarbonyl)amino)acetamido)-2-phenylethyl (1R,2S,5R)-2-isopropyl-5methylcyclohexane-1-carboxylate

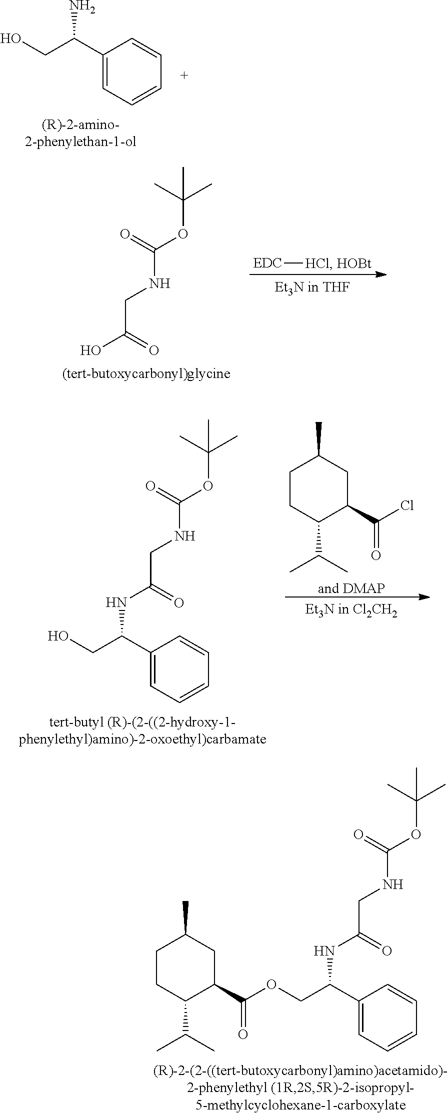

Step 1—A 500 mL round-bottom flask (A) was charged with a magnetic stir bar, 30 mL of tetrahydrofuran (THF), (R)-2-amino-2-phenylethan-1-ol (1.02 g, 7.4 mmol), N-(3-dimethylaminopropyl)-N′-ethylcarbodiimide hydrochloride (1.60 g EDC-HCl, 8.3 mmol), triethylamine (4.92 g Et₃N, 48.6 mmol) and 1H-benzo[d][1,2,3] triazol-1-ol (0.85 g HOBt, 6.3 mmol). A separate 100 mL round-bottom flask (B) was charged with tert-butoxycarbonyl)glycine (1.19 g BOC-Gly-OH, 6.8 mmol) and 40 mL of methylene chloride (CH₂Cl₂). The contents of flask B were transferred into flask A by pipette. Flask B was rinsed with two additional 10 mL aliquots of methylene chloride. These were added to flask A. The reaction vessel was closed with a septum and the heterogeneous mixture was mixed at 800 r.p.m. and 20-25° C. After 22 hours the heterogeneous reaction mixture was transferred to a one liter separatory funnel. The reaction flask was rinsed with 2×25 mL portions of THF. These were added to the separatory funnel. The separatory funnel contained 50 mL of ethyl acetate (EtOAc) and 50 mL of water. The reaction products were observed to dissolve completely upon addition to the funnel. The water phase was extracted with 2×25 mL aliquots of EtOAc. The organic phases were recovered and combined. The combined organic phase was subsequently extracted with 1 N HCl (3×40 mL), saturated sodium bicarbonate (2×20 mL), and saturated potassium chloride (2×20 mL). The recovered organic layer was dried over anhydrous sodium sulfate. The organic phase was filtered through Whatman #4 filter to remove the drying agent. The solvent was removed in vacuo (45-50° C.) to give 0.55 g of a light amber colored and sticky residue, tert-butyl (R)-(2-((2-hydroxy-1-phenylethyl)amino)-2-oxoethyl)carbamate: MS (ESI) m/z 295 (MH⁺).

Step 2—A 100 mL round-bottom flask (A) was charged with a stir bar, tert-butyl (R)-(2-((2-hydroxy-1-phenylethyl)amino)-2-oxoethyl)carbamate (0.53 g, 1.8 mmol), 4-(dimethylamino)pyridine (0.045 g DMAP, 0.4 mmol) dissolved in 10 mL of methylene chloride, triethylamine (0.90 g TEA, 8.9 mmol) dissolved in 10 mL of methylene chloride and (1R,2S,5R)-2-isopropyl-5-methylcyclohexane-1-carbonyl chloride (0.32 g, 1.6 mmol) dissolved in 10 mL of methylene chloride while purging the flask headspace with dry nitrogen. The glassware was rinsed with 10 mL of methylene chloride which was added to the reaction flask. The flask was closed with a septum and immersed in an ice bath with magnetic mixing set to 250 r.p.m. and allowed to react under static nitrogen atmosphere and melting ice bath/20-25° C. for 20 hours. The reactor contents were transferred to a 250 mL separatory funnel following the reaction period. The organic phase was extracted with 1.0 M HCl (3×50 mL), saturated sodium bicarbonate (2×50 mL) and saturated potassium chloride (1×50 mL). The recovered organic layer was dried over anhydrous sodium sulfate. The organic phase was filtered through Whatman #4 filter to remove the drying agent. The solvent was removed in vacuo (45-50° C.) to give approximately 0.6 g of crude product. The crude product was purified by flash chromatography in which the stationary phase was a 6 inch column, 25 mm diameter of Silica Gel 60 and the mobile phase was 65:35 v/v hexanes and ethyl acetate to give 0.4 g of the target compound, (R)-2-(2-((tert butoxycarbonyl)amino)acetamido)-2-phenylethyl (1R,2S,5R)-2-isopropyl-5-methylcyclohexane-1-carboxylate as an off-white solid: MS (ESI) m/z 461 (MH⁺).

### Example 26

### (R)-2-(2-aminoacetamido)-2-phenylethyl (1R,2S,5R)-2-isopropyl-5-methylcyclohexane-1-carboxylate hydrochloride

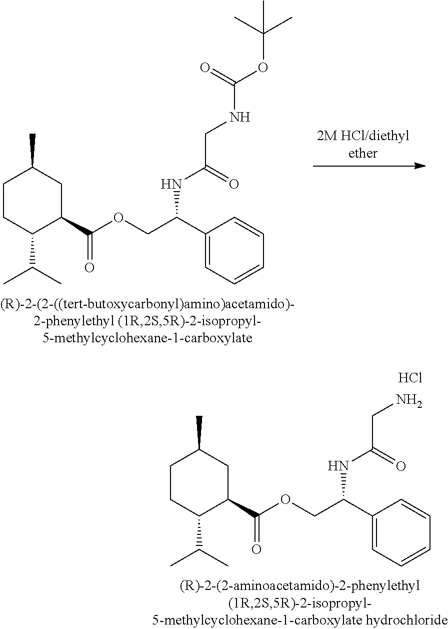 [SMILES: CC(C)[C@@H]1CC[C@@H](C)C[C@H]1C(=O)OC[C@H](NC(=O)CNC(=O)OC(C)(C)C)c1ccccc1 ; Cl ; CC(C)[C@@H]1CC[C@@H](C)C[C@H]1C(=O)OC[C@H](NC(=O)CN)c1ccccc1]

A 100 mL round-bottom flask was charged with a magnetic stir bar, (R)-2-(2-((tert-butoxycarbonyl)amino)acetamido)-2-phenylethyl (1R,2S,5R)-2-isopropyl-5-methylcyclohexane-1-carboxylate (0.3 g, 0.7 mmol), 5 mL of methylene chloride and 50 mL of 2.0 M HCl dissolved in diethyl ether (100 mmol HCl). The reaction vessel was closed with a septum and connected to an oil bubbler. The reactor contents were stirred at 150 r.p.m. under a static nitrogen atmosphere and 20-25° C. Thin layer chromatography indicated that the reaction was still incomplete after 3 hours. An additional 50 mL of 2.0 M HCl dissolved in diethyl ether (100 mmol HCl) was added to the flask. The reaction was allowed to continue to mix for a total of 24 hours. The diethyl ether solvent and any volatile reaction by-products were removed in vacuo (40-45° C.) to give 0.25 g of the title compound as an off-white solid: MS (ESI) m/z 361 (MH⁺ of the free amine)

### Example 27

### Synthesis of (S)-2-(((1R,2S,5R)-2-isopropyl-5-methylcyclohexyl)oxy)-2-oxo-1-phenylethan-1-aluminum chloride

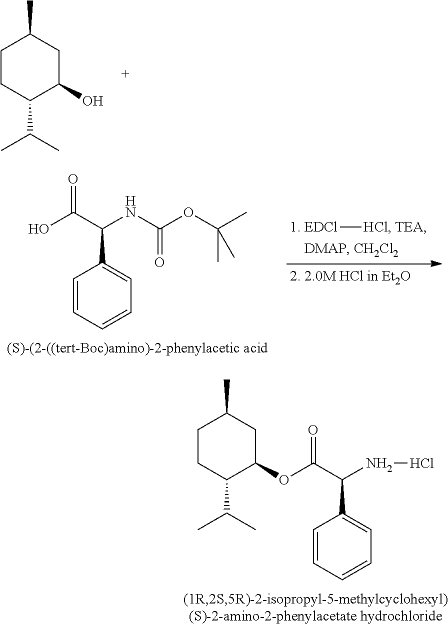

Step 1. In a flame-dried 500 mL round-bottomed flask equipped with a stir bar and a N₂ inlet was added anhydrous CH₂Cl₂ (200 mL) and (1R,2S,5R)-(−)-Menthol (CAS #2215-51-5, 2.82 g). The solution was stirred 15 minutes and then 4-dimethylaminopyridine (CAS #1122-58-3, 245 mg), triethylamine (CAS #121-44-8, 9.2 mL), N,N-dimethyl-N-(ethylcarbonimidoyl)-1,3-propanediamine hydrochloride (CAS #25952-53-8, 3.8 g) and (S)-2-((tert-butoxycarbonyl)amino)-2-phenylacetic acid (S-Boc-Phg-OH) (CAS #2900-27-8, 5 g) were added sequentially over 10 minutes. The reaction was stirred for 24 h and then quenched with 1 N HCl. The layers were separated with a separatory funnel and the organic layer was collected, washed with 1N HCl (3×100 mL) and brine (300 mL). The organic layer was then separated and dried over anhydrous Na₂SO₄. After filtration the solvent was removed on a rotary evaporator (5-10 mm Hg) to provide a white solid which was used in step 2 without further purification.

Step 2. The solid was dissolved in Et₂O (200 mL) and 2M HCl (10 mL) was added via addition funnel. The reaction was stirred 24 hours, at which point a precipitate had formed. The reaction was rotovapped directly to dryness at that point (5-10 mm Hg) and the resulting solid was triturated with Et₂O (3×200 mL) and then filtered through a Buchner funnel. The resulting white solid was dried 24 hours under vacuum (5-10 mm Hg) to achieve constant weight: yield: 500 mg. LC/MS: m/z 290 (M⁺ of free amine)

### Example 28

### Synthesis of 2-(((S)-2-(((1R,2S,5R)-2-isopropyl-5-methylcyclohexyl)oxy)-2-oxo-1-phenylethyl)amino)-2-oxoethan-1-aluminum chloride

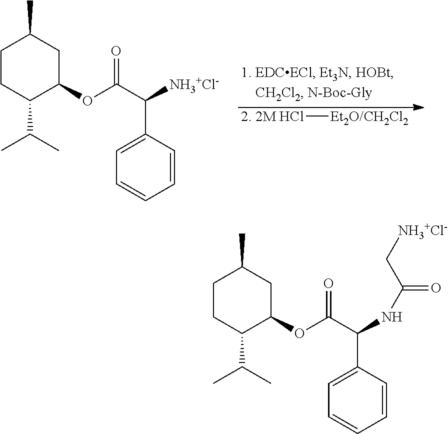

Step 1. A 250 mL 3-neck round bottom flask equipped with a condenser with an outlet to a Firestone valve (positive nitrogen pressure) and a magnetic stir bar was charged with Boc-Gly (0.34 g, 1.90 mmol), HOBt (0.26 g, 1.90 mmol), EDC-HCl (0.36 g, 1.90 mmol), and 95 mL anhydrous CH₂Cl₂. The solution was stirred under nitrogen and triethylamine (530 μL, 3.84 mmol) was added. To the heterogeneous mixture was added (S)-2-(((1R,2S,5R)-2-isopropyl-5-methylcyclohexyl)oxy)-2-oxo-1-phenylethan-1-aluminum chloride (0.500 g, 1.54 mmol) dissolved in 20 mL of CH₂Cl₂. The heterogeneous mixture was stirred under a nitrogen atmosphere for 24 hours. The reaction mixture was transferred to a 1L separatory funnel containing CH₂Cl₂ (100 mL) and water (100 mL). The aqueous layer was separated and extracted again with 2×80 mL of CH₂Cl₂. The combined organic phases were washed with 1N HCl solution (2×50 mL), H₂O (1×50 mL), saturated sodium bicarbonate solution (3×50 mL), and brine (1×50 mL). The solution was dried over anhydrous sodium sulfate, filtered, and concentrated under vacuum at 38° C. to give 400 mg of white solid, which was used in step 2.

Step 2. The solid was dissolved in CH₂Cl₂ (50 mL) and 2M HCl/Et₂O was added (10 mL). The reaction was stirred 24 hours, and then the solvents were stripped off in-vacuo (5-10 mm Hg). The resulting white solid was triturated with Et₂O (3×100 mL) and then filtered and dried 24 hours under vacuum (5-10 mm Hg). Yield: 210 mg. MS (ESI) m/z 381 (negative ion, M−1, 100%).

Using the procedure described above and the appropriately protected amino acid, the following compounds can be synthesized by those skilled in the art:
(2S)-1-(2-(((1R,2S,5R)-2-isopropyl-5-methylcyclohexyl)oxy)-2-oxo-1-phenylethyl)amino)-1-oxopropan-2-aluminum chloride.
(2R)-1-((2-(((1R,2S,5R)-2-isopropyl-5-methylcyclohexyl)oxy)-2-oxo-1-phenylethyl)amino)-1-oxopropan-2-aluminum chloride.

### Example 29

### ((1R,2S,5R)-2-isopropyl-5-methylcyclohexyl)methyl 2-amino-2-phenylacetate hydrochloride

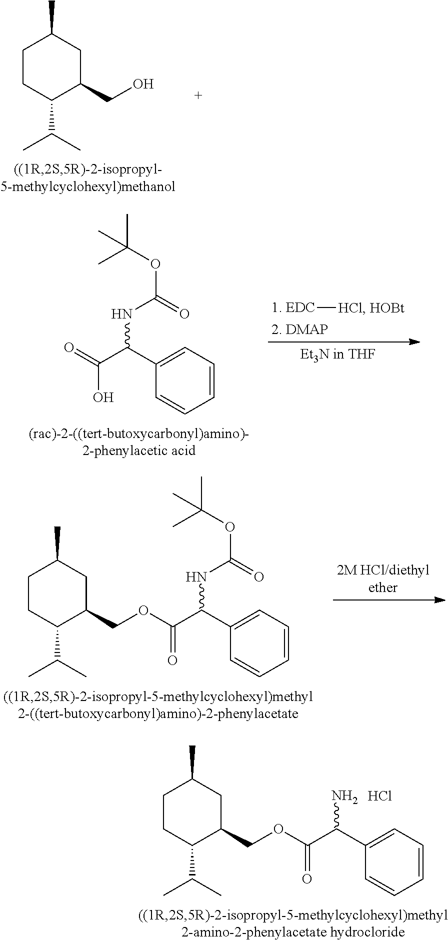

Step 1—A 500 mL round-bottom flask (A) was charged with a magnetic stir bar, N-(3-dimethylaminopropyl)-N′-ethylcarbodiimide hydrochloride (2.71 grams EDC-HCl, 14.1 mmol) dissolved in 30 mL of tetrahydrofuran (THF), triethylamine (2.97 grams Et₃N, 29.3 mmol) dissolved in 15 mL of THF, 1H-benzo[d][1,2,3] triazol-1-ol (1.99 grams HOBt, 14.7 mmol) dissolved in 20 mL of THF and (racemic)-2-((tert-butoxycarbonyl)amino)-2-phenylacetic acid (3.23 grams, N-Boc-DL-phenylglycine [3601-66-9], 12.8 mmol) dissolved in 20 mL of THF. A separate 100 mL round-bottom flask (B) was charged with ((1R,2S,5R)-2-isopropyl-5-methylcyclohexyl) methanol (2.05 grams, 12.0 mmol, prepared according to Xiangtan Daxue Ziran Kexue Xuebao (2009), 31(3), 96-100) and 20 mL of THF. The contents of flask B were transferred into flask A by pipette. Flask B was rinsed with two additional 5 mL aliquots of THF. These were added to flask A. The reaction vessel was closed with a septum and the heterogeneous mixture was mixed at 500 r.p.m. under static argon atmosphere and 20-25° C. for 20 hours. Thin layer chromatography (TLC) indicated that the reaction was incomplete following the 20 hour reaction period. To try to push the reaction 4-(dimethylamino)pyridine (0.68 grams DMAP, 5.6 mmol) dissolved in 10 mL of THF was added to reaction flask A. The reactor contents were allowed to continue to mix for an additional 4 hours. TLC indicated that the reaction was still incomplete after 27 hours of total reaction time. Additional coupling reagents were added to the reaction: N-(3-dimethylaminopropyl)-N′-ethylcarbodiimide hydrochloride (1.05 grams EDC-HCl, 5.5 mmol) dissolved in 10 mL of (THF) and 4-(dimethylamino)pyridine (0.84 grams DMAP, 6.9 mmol) dissolved in 10 mL of THF, and the glassware was rinsed with two 10 mL aliquots of THF which were also added to reaction flask A. The reactor contents were allowed to continue to mix overnight. TLC indicated that the reaction was complete after 44 hours of total reaction time. The heterogeneous reaction mixture was transferred to a one liter separatory funnel. The reaction flask was rinsed with 40 mL of THF which was added to the separatory funnel containing 50 mL of ethyl acetate (EtOAc) and 100 mL of water. The heterogeneous reaction products were observed to dissolve completely upon addition to the separatory funnel. The water phase was extracted with 40 mL of EtOAc. The ethyl acetate was recovered and combined with the organic phase. The combined organic phases were subsequently extracted with 120 mL of 1 N HCl, 90 mL of saturated sodium bicarbonate and 60 mL of saturated potassium chloride. The recovered organic layer was dried over anhydrous sodium sulfate. The organic phase was filtered through Whatman #4 filter to remove the drying agent. The solvent was removed in vacuo (35-40° C.) to give approximately 4.6 g of a light yellow/amber colored residue, ((1R,2S,5R)-2-isopropyl-5-methylcyclohexyl)methyl 2-((tert-butoxycarbonyl)amino)-2-phenylacetate. MS (ESI) m/z 404 (MH⁺).

Step 2—A 500 mL round-bottom flask was charged with a magnetic stir bar, ((1R,2S,5R)-2-isopropyl-5-methylcyclohexyl)methyl 2-((tert-butoxycarbonyl)amino)-2-phenylacetate (3.42 g, 8.5 mmol), 5 mL of diethyl ether and 3×33 mL portions of 2.0 M HCl dissolved in diethyl ether (198 mmol HCl). The reaction vessel was closed with a septum, the headspace purged with argon and the apparatus connected to an oil bubbler. The heterogeneous reactor contents were stirred at 200 r.p.m. and 20-25° C. The reactor contents were allowed to continue to mix and TLC indicated that the reaction was incomplete after a 21 hour reaction period. The spent diethyl ether/HCl reagent was removed in vacuo and 100 mL of fresh 2.0 M HCl dissolved in diethyl ether (200 mmol HCl) was added to the flask. The reaction was allowed to continue to mix for an additional 22 hours (i.e. total reaction time of 43 hours). TLC indicated that the reaction was complete following the second addition of reagent. The heterogeneous reaction products were filtered through a Whatman #4 filter and the recovered solids were washed with 3×50 mL portions of diethyl ether to give 2.41 g of the title compound as an off-white solid. MS (ESI) m/z 304 (MH⁺ of the free amine).

### Example 30

### ((1R,2S,5R)-2-isopropyl-5-methylcyclohexyl)methyl 2-(2-aminoacetamido)-2-phenylacetate hydrochloride

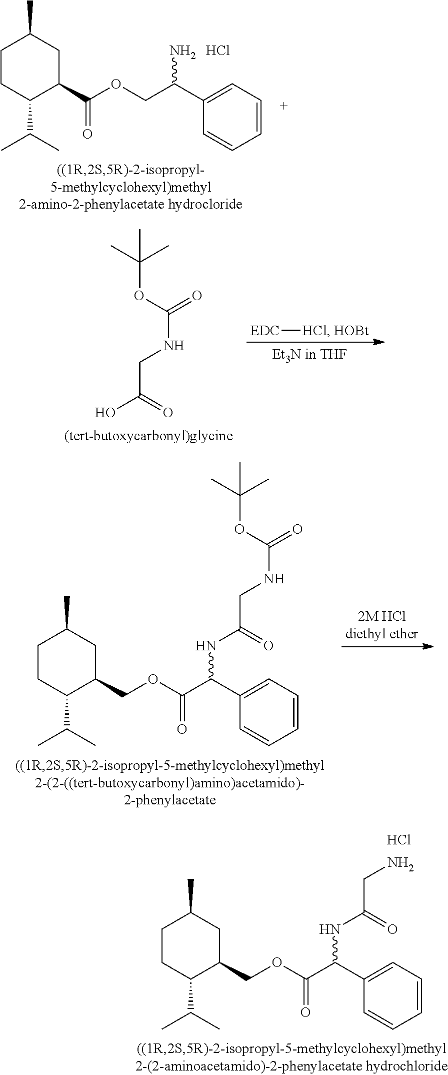

Step 1. A 100 mL round-bottom flask was charged with a magnetic stir bar, 10 mL of tetrahydrofuran (THF), ((1R,2S,5R)-2-isopropyl-5-methylcyclohexyl)methyl 2-amino-2-phenylacetate hydrochloride (0.5 g, 1.5 mmol), (tert-butoxycarbonyl)glycine (0.27 g Boc-Gly-OH, 1.5 mmol), triethylamine (0.47 g Et₃N, 4.6 mmol), 1H-benzo[d][1,2,3] triazol-1-ol (0.25 g HOBt, 1.8 mmol) dissolved in 10 mL of THF and N-(3-dimethylaminopropyl)-N′-ethylcarbodiimide hydrochloride (0.33 grams EDC-HCl, 1.7 mmol) along with an additional 15 mL of THF to rinse weighing containers. The reaction vessel was closed with a septum and the heterogeneous mixture was mixed at 700 r.p.m. under static argon atmosphere at 20-25° C. for 22 hours. The heterogeneous reaction mixture was transferred to a 250 mL separatory funnel. The reaction flask was rinsed with 15 mL of THF and then 40 mL of water. These rinses were added to the separatory funnel. An additional 25 mL of ethyl acetate (EtOAc) was added to the separatory funnel. The water phase was extracted with 20 mL of EtOAc. The combined organic phases were subsequently extracted with 55 mL of 1 N HCl, 40 mL of saturated sodium bicarbonate and 20 mL of saturated potassium chloride and dried over anhydrous sodium sulfate. The organic phase was filtered through Whatman #4 filter to remove the drying agent. The solvent was removed in vacuo (35-40° C.) to give 0.66 g of residue of the intermediate product, ((1R,2S,5R)-2-isopropyl-5-methylcyclohexyl)methyl 2-(2-((tert-butoxycarbonyl)amino)acetamido)-2-phenylacetate. MS (ESI) m/z 461 (MH⁺).

Step 2. A 100 mL round-bottom flask was charged with a magnetic stir bar, ((1R,2S,5R)-2-isopropyl-5-methylcyclohexylmethyl 2-(2-((tert-butoxycarbonyl)amino)acetamido)-2-phenylacetate (0.62 grams, 1.3 mmol) and 50 mL of 2.0 M HCl dissolved in diethyl ether (100 mmol HCl). The reaction vessel was closed with a septum, the headspace purged with argon and the apparatus connected to an oil bubbler. The reactor contents were stirred at 200 r.p.m. at 20-25° C. for 18 hours. Thin layer chromatography indicated that the reaction was still incomplete after the 18 hour reaction period. The spent diethyl ether/HCl reagent was removed in vacuo and 50 mL of fresh 2.0 M HCl dissolved in diethyl ether (100 mmol HCl) was added to the flask. The reaction was allowed to continue to mix for an additional 18 hours (i.e. total reaction time of 42 hours). The reaction mixture was filtered through a Whatman #4 filter. The diethyl ether/HCl reagent was removed in vacuo to give 0.41 g of the title compound which as an off-white solid: MS (ESI) m/z 361 (MH⁺ of the free amine)

### Example 31

### (S)-2-hydroxy-2-phenylethyl (1R,2S,5R)-2-isopropyl-5-methylcyclohexane-1-carboxylate

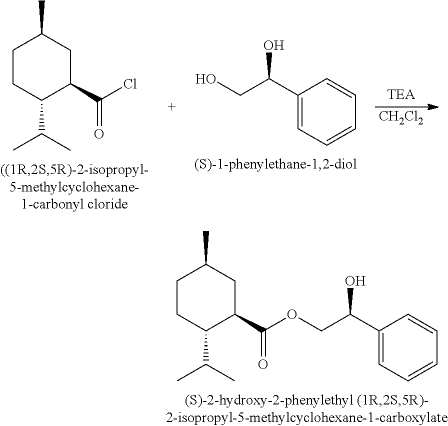

A 100 mL round-bottom flask (A) was charged with a magnetic stir bar, (S)-1-phenylethane-1,2-diol (1.11 g (8.0 mmol), 10 mL of methylene chloride (CH₂Cl₂) and triethylamine (1.95 g TEA, 19.3 mmol) dissolved in 10 mL of CH₂Cl₂. Flask A was placed in an ice bath to equilibrate and the mixing speed was set to 250 r.p.m. A separate, 50 mL round-bottom flask (B) was charged with (1R,2S,5R)-2-isopropyl-5-methylcyclohexane-1-carbonyl chloride (1.84 g L-Men-AC, 9.1 mmol) and 40 mL of CH₂Cl₂. The solution in flask B was transferred poured into a 60 mL pressure-equalizing addition funnel. The funnel was connected to the flask. The assembled reaction apparatus was connected to an oil bubbler and the headspace purged with dry nitrogen. The solution in the addition funnel was added to the reaction flask A over 16 minutes while mixing at 500 r.p.m. The reaction contents were allowed to mix at 250 r.p.m. under static nitrogen atmosphere and melting ice bath/20-25° C. for 44 hours. The reaction mixture was transferred to a 250 mL separatory funnel following the reaction period. The organic phase was extracted with 1 N HCl (3×25 mL), 1 N NaOH (1×25 mL), and saturated sodium chloride (2×25 mL). The recovered organic layer was dried over anhydrous sodium sulfate. The organic phase was filtered through Whatman #4 filter to remove the drying agent. The solvent was removed in vacuo (35-40° C.) to give approximately 2 grams of a light amber colored liquid. The crude product was purified by flash chromatography (Silica Gel 60 9 inch×85 mm column) eluting with 80:20 v/v hexanes and ethyl acetate to give approximately 0.7 g of the title compound, (S)-2-hydroxy-2-phenylethyl (1R,2S,5R)-2-isopropyl-5-methylcyclohexane-1-carboxylate, as a light yellow liquid: MS (ESI) m/z 305 (MH⁺).

### Example 32

### (S)-2-hydroxy-2-phenylethyl (1R,2S,5R)-2-isopropyl-5-methylcyclohexane-1-carboxylate

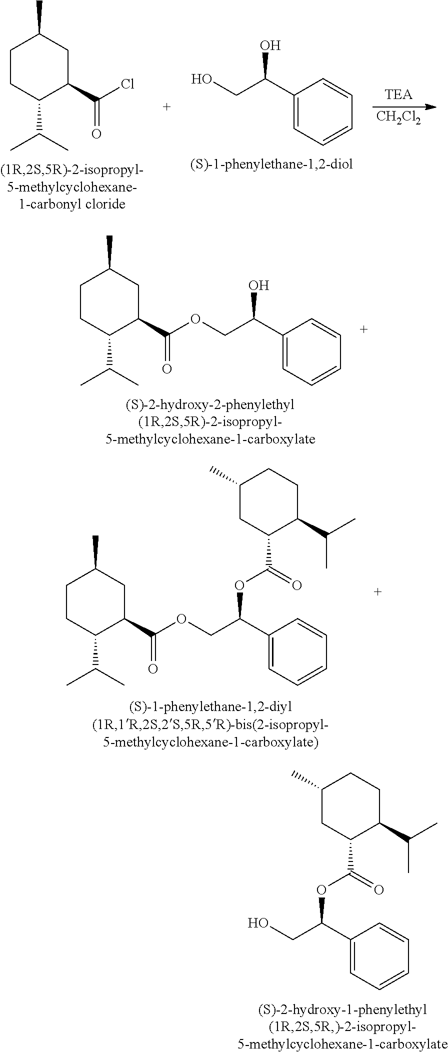

A 100 mL round-bottom flask (A) was charged with a magnetic stir bar, (S)-1-phenylethane-1,2-diol (1.11 g, 8.0 mmol), 10 mL of methylene chloride (CH₂Cl₂) and triethylamine (1.95 g TEA, 19.3 mmol) dissolved in 10 mL of CH₂Cl₂. Flask A was placed in an ice bath to equilibrate and the mixing speed was set to 250 r.p.m. A separate, 50 mL round-bottom flask (B) was charged with (1R,2S,5R)-2-isopropyl-5-methylcyclohexane-1-carbonyl chloride (1.84 grams, 9.1 mmol) and 40 mL of CH₂Cl₂. The solution in flask B was transferred into a 60 mL, pressure-equalizing addition funnel. The funnel was connected to the flask. The assembled reaction apparatus was connected to an oil bubbler and the headspace purged with dry nitrogen. The acid chloride solution in the addition funnel was added to the reaction flask A over 16 minutes while mixing at 500 r.p.m. The reaction contents were allowed to mix at 250 r.p.m. under static nitrogen atmosphere and melting ice bath/20-25° C. for 44 hours. The reaction mixture was transferred to a 250 mL separatory funnel following the reaction period. The organic phase was extracted with 1 N HCl (3×25 mL), 1 N NaOH (1×25 mL), and saturated sodium chloride (2×25 mL). The recovered organic layer was dried over anhydrous sodium sulfate. The organic phase was filtered through a Whatman #4 filter to remove the drying agent. The solvent was removed in vacuo (35-40° C.) to give approximately 2 g of a light amber colored liquid. The crude product was separated by flash chromatography. The stationary phase was a 9 inch long column, 85 mm in diameter of Silica Gel 60 and the mobile phase was 80:20 v/v hexanes/ethyl acetate. The mixed solvent was able to resolve the reaction mixture into three main reaction products having R_{f} values of 0.19, 0.29 and 0.58.
Approximately 0.7 g of the title compound, (S)-2-hydroxy-2-phenylethyl (1R,2S,5R)-2-isopropyl-5-methylcyclohexane-1-carboxylate was recovered as a clear and viscous oil. The R_{f} for this compound was 0.29: MS (ESI) m/z 305 (MH⁺).
Approximately 0.6 g of diester, (S)-1-phenylethane-1,2-diyl (1R,1′R,2S,2′S,5R,5′R)-bis(2-isopropyl-5-methylcyclohexane-1-carboxylate), was recovered as a clear and viscous oil. The R_{f} for this compound was 0.58: MS (ESI) m/z 287 (C₁₉H₂₇O⁺, 100%), m/z 139 (C₁₀H₁₉⁺, 63%) and m/z 167 (C₁₁H₁₉O⁺, 12%).
Approximately 0.04 g of (S)-2-hydroxy-1-phenylethyl (1R,2S,5R)-2-isopropyl-5-methylcyclohexane-1-carboxylate was recovered as a light amber residue. The R_{f} for this compound was 0.19: MS (ESI) m/z 305 (MH⁺).

### Example 33

### (S)-2-(((tert-butoxycarbonyl)glycyl)oxy)-2-phenylethyl (1R,2S,5R)-2-isopropyl-5-methylcyclohexane-1-carboxylate

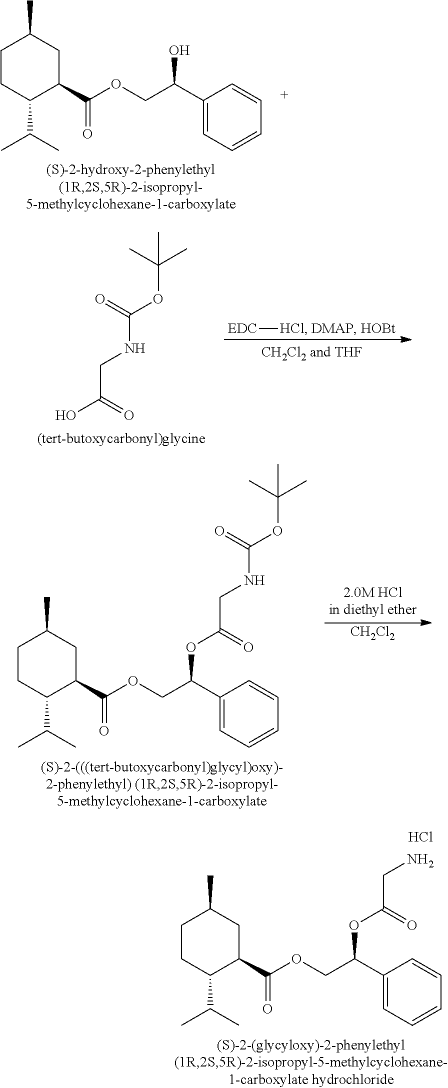

Step 1—A 50 mL, round-bottom flask was charged with 0.3 g (0.97 mmols) of the starting (S)-2-hydroxy-2-phenylethyl (1R,2S,5R)-2-isopropyl-5-methylcyclohexane-1-carboxylate and 2 mL of dichloromethane (CH₂Cl₂). A separate vial was charged with 0.2 g (1.12 mmols) of (tert-butoxycarbonyl)glycine (Boc-Gly-OH) and 2 mL of CH₂Cl₂. A separate vial was charged with 0.26 g (1.34 mmols) of 3-(((ethylimino)methylene)amino)-N,N-dimethylpropan-1-amine hydrochloride (EDC-HCl) and 2 mL of CH₂Cl₂. The EDC-HCl was not soluble in the CH₂Cl₂. A separate vial was charged with 0.14 g (1.01 mmols) of 1H-benzo[d][1,2,3]triazol-1-ol (HOBt) and 3 mL of tetrahydrofuran. The HOBt was not completely soluble in the tetrahydrofuran. A separate vial was charged with 0.26 g (2.24 mmols) of 4-dimethylamino pyridine (DMAP) and 3 mL of dichloromethane (CH₂Cl₂). The DMAP solution was then added to the EDC-HCl slurry. This resulted in a homogenous solution of DMAP and EDC-HCl. Then the Boc-Gly-OH solution and the HOBt mixture were both added to the solution of L-Men-ester-(S)-2PhE-2-ol in the reaction flask. Finally, the DMAP/EDC-HCl solution was poured into the reaction flask which was then closed with a septum. The head space was purged with dry nitrogen and the reactor was connected to an oil bubbler. The reaction was allowed to mix at 160 r.p.m. and 20-25° C. under static nitrogen atmosphere for 90 hours. The reaction was incomplete after 90 hours. Therefore, an additional 0.28 g (1.45 mmols) of EDC-HCl dispersed in 70:30 v/v CH₂Cl₂ and tetrahydrofuran was added to the reaction flask. The reaction was allowed to continue to mix for an additional 24 hours and determined to be complete following the second addition of a of EDC-HCl and a total reaction time of 115 hours. The reaction mixture was poured into a 250 mL separatory funnel and the organic layer was extracted with three×50 mL aliquots of 1.0 N HCl, two×25 mL aliquots of 1.0 N NaOH and one 25 mL aliquot of saturated sodium chloride. The extracted organic layer was dried over anhydrous sodium sulfate overnight and then filtered through a Whatman #4 filter to remove the drying agent. The solvent was removed in vacuo to give 0.38 g of the intermediate, (S)-2-(((tert-butoxycarbonyl)glycyl)oxy)-2-phenylethyl (1R,2S,5R)-2-isopropyl-5-methylcyclohexane-1-carboxylate which was a clear and viscous residue having almost no color: MS (ESI) m/z 462 (MH⁺).

Step 2—(S)-2-(glycyloxy)-2-phenylethyl (1R,2S,5R)-2-isopropyl-5-methylcyclohexane-1-carboxylate hydrochloride

A 100 mL, round-bottom flask was charged with 0.29 g (0.63 mmols) of the starting (S)-2-(((tert-butoxycarbonyl)glycyl)oxy)-2-phenylethyl (1R,2S,5R)-2-isopropyl-5-methylcyclohexane-1-carboxylate, 2 mL of CH₂Cl₂ and a magnetic stir bar. Then 50 mL of 2.0 M HCl (100 mmols) in diethyl ether was poured into the reaction flask. The flask was closed with a septum and connected to an oil bubbler. The reactor contents were allowed to mix at 250 r.p.m. and 20-25° C. for 21 hours during which time solids were observed to precipitate from solution. The reaction slurry was filtered through a Whatman, 934-AH, glass micro-fiber filter. The recovered solids were then washed with diethyl ether followed by drying in vacuo to give 0.09 g of the title compound (S)-2-(glycyloxy)-2-phenylethyl (1R,2S,5R)-2-isopropyl-5-methylcyclohexane-1-carboxylate hydrochloride. MS (ESI) m/z 362 (MH⁺ of the free amine).

### Example 34

### EC50 Analysis of Sensate Analogs Using TRPM8 Activation

It is now well established that sensations such as cool or cold can be attributed to activation of receptors at peripheral nerve fibers by a stimulus such as low temperature or a chemical coolant, which produces electrochemical signals that travel to the brain that then interprets, organizes and integrates the incoming signals into a perception or sensation. Different classes of receptors have been implicated in sensing cold temperatures or chemical coolant stimuli at mammalian sensory nerve fibers. Among these receptors, a major candidate involved in sensing cold has been identified and designated as cold- and menthol-sensitive receptor CMR1, also known as TRPM8. The TRPM8 nomenclature for the receptor comes from its characterization as a non-selective cation channel of the transient receptor potential (TRP) family, which is activated by stimuli including low temperatures, menthol and other chemical coolants. The TRPM8 receptor is provided as SEQ ID NO: 3. However, the precise mechanisms underlying the perception of a pleasant cooling sensation on skin or oral surfaces are presently not clearly understood. While it has been demonstrated that the TRPM8 receptor is activated by menthol and other coolants, other receptors, such as TRPA1 (SEQ ID NO: 2) and TRPV1 (SEQ ID NO: 1), have been shown to influence the cooling response and duration. However, there are likely more receptors involved and it is not fully understood what other receptors may participate in the perception of cooling, and to what extent these receptors need to be stimulated or perhaps suppressed in order for the overall perceived sensation to be pleasant, cooling and refreshing. For example, menthol is widely used as a cooling agent, but menthol can also produce other sensations including tingling, burning, prickling and stinging as well as a minty smell and bitter taste. Thus, it can be inferred that menthol acts on many different receptors, including cold, warm, pain and taste receptors.

To determine what effect, if any, test compounds (shown in TABLE 1) had on TRPM8 (SEQ ID NO: 3) activation the protocol listed below was used.

### TRPM8 Protocol-FLIPR Assay

To determine whether TRPM8 is activated, the intracellular calcium ion (Ca²⁺) level was measured from transfected cells with the TRPM8 receptor sequence (SEQ ID NO: 3). HEK-293 (human embryonic kidney) cells stably transfected with human TRPM8 were grown in 15 ml growth medium (high glucose DMEM (Dulbecco's Modification of Eagle's Medium) supplemented with 10% FBS (fetal bovine serum), 100 ug/ml penicillin/streptomycin, 5 μg/m1 blasticindin, and 100 μg/ml zeocin) in a 75 cm² flask for 3 days at 37° C. in a mammalian cell culture incubator set at 5% CO₂. Cells were detached with addition of 2 ml of trypsin-EDTA buffer (GIBCO® 25200, Invitrogen, Grand Island, N.Y.) for about 2-3 mM. Trypsin was inactivated by addition of 8 ml growth medium. Cells were transferred to a 50 ml tube and centrifuged at 850 rpm for 3 minutes to remove medium. After centrifugation, a pellet of cells was formed in the bottom of the tube separating them from the supernatant solution. The supernatant was discarded and the cell pellet was suspended in 1 ml of fresh growth medium to which 5 μl (12.5 μg) of Fluo-4 AM (Molecular Probes, Inc., Eugene, Oreg.) calcium indicator was added and incubated for 30 mM with gentle shaking. Fluo-4 AM is a fluorescent dye used for quantifying cellular Ca²⁺ concentrations in the 100 nM to 1 microM range. At the end of 30 minutes, 45 ml of assay buffer (1×BSS (Hank's Balanced Salt Solution), 20 mM HEPES (4-(2-Hydroxyethyl)-1-piperazineethanesulfonic acid)) was added to wash cells and the resulting mixture was then centrifuged at 850 rpm for 3 minutes to remove excess buffer and Fluo-4 AM calcium indicator.

The pelleted cells were re-suspended in 10 ml assay buffer and 90 μl aliquots (˜50,000 cells) per well delivered to a 96-well assay plate containing 10 μl of test compounds (1 mM in assay buffer, final concentration 100 μM) or buffer control and incubated at room temperature for 30 minutes. After 30 minutes, a plate was placed into a fluorometric imaging plate reader (FLIPR384 from Molecular Devices, Sunnyvale, Calif.) and basal fluorescence recorded (excitation wave length 488 nm and emission wave length 510 nm). Then 20 μl of 100 mM of TRPM8 agonist WS5 coolant in the assay buffer was added and fluorescence recorded. For determining the direct effect of test compounds on TRPM8, fluorescence was measured immediately after addition of each compound (TABLE 1). Additional discussion of the FLIPR method can be found in Smart et al., Characterization using FLIPR of human vanilloid VR1 receptor pharmacology, European Journal of Pharmacology 417, 51-58 (2001) and Liu et al., Development and validation of a platelet calcium flux assay using a fluorescent imaging plate reader, Analytical Biochemistry 357, 216-224 (2006).

TABLE 1

| Sample # | Molecule | TRPM8 EC 50 Value | |
|---|---|---|---|
| #1 | 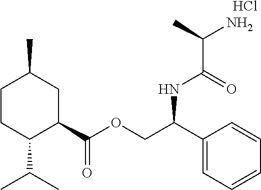 [SMILES: Cl ; CC(C)[C@@H]1CC[C@@H](C)C[C@H]1C(=O)OC[C@@H](NC(=O)[C@@H](C)N)c1ccccc1] | 0.008076 | micro Molar |
| #2 | 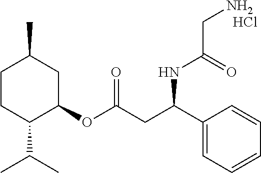 [SMILES: CC(C)[C@@H]1CC[C@@H](C)C[C@H]1OC(=O)C[C@@H](NC(=O)CN)c1ccccc1 ; Cl] | 0.062 | micro Molar |
| #3 | 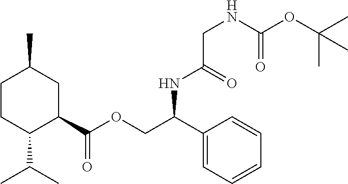 [SMILES: CC(C)[C@@H]1CC[C@@H](C)C[C@H]1C(=O)OC[C@@H](NC(=O)CNC(=O)OC(C)(C)C)c1ccccc1] | 23.2 | micro Molar |
| #4 | 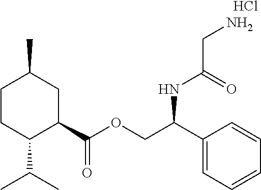 [SMILES: Cl ; CC(C)[C@@H]1CC[C@@H](C)C[C@H]1C(=O)OC[C@@H](NC(=O)CN)c1ccccc1] | 0.064 | micro Molar |
| #5 | 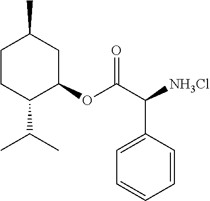 | 60.2 | micro Molar |
| #6 | 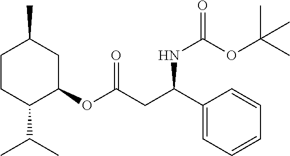 [SMILES: CC(C)[C@@H]1CC[C@@H](C)C[C@H]1OC(=O)C[C@@H](NC(=O)OC(C)(C)C)c1ccccc1] | Too high to measure | |
| #7 | 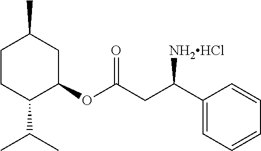 | 8 | micro Molar |
| #8 | 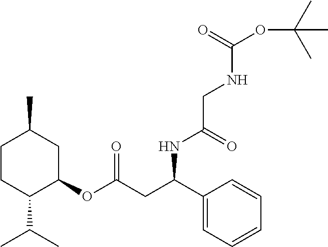 [SMILES: CC(C)[C@@H]1CC[C@@H](C)C[C@H]1OC(=O)C[C@@H](NC(=O)CNC(=O)OC(C)(C)C)c1ccccc1] | Too high to measure | |
| #9 | 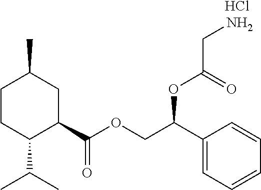 [SMILES: Cl ; CC(C)[C@@H]1CC[C@@H](C)C[C@H]1C(=O)OC[C@@H](OC(=O)CN)c1ccccc1] | 0.013 | micro Molar |
| #10 | 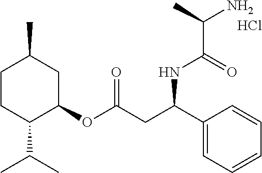 [SMILES: CC(C)[C@@H]1CC[C@@H](C)C[C@H]1OC(=O)C[C@@H](NC(=O)[C@@H](C)N)c1ccccc1 ; Cl] | 0.03675 | micro Molar |
| #11 | 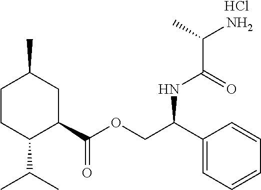 [SMILES: Cl ; CC(C)[C@@H]1CC[C@@H](C)C[C@H]1C(=O)OC[C@@H](NC(=O)[C@H](C)N)c1ccccc1] | 0.3426 | micro Molar |
| #12 | 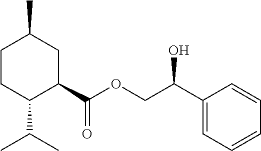 [SMILES: CC(C)[C@@H]1CC[C@@H](C)C[C@H]1C(=O)OC[C@@H](O)c1ccccc1] | 0.59 | micro Molar |
| #13 | 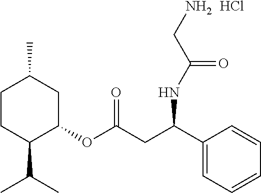 [SMILES: Cl ; CC(C)[C@H]1CC[C@H](C)C[C@@H]1OC(=O)C[C@@H](NC(=O)CN)c1ccccc1] | 0.8822 | micro Molar |
| #14 | 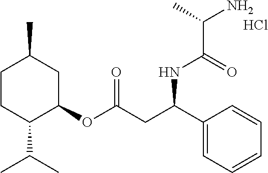 [SMILES: CC(C)[C@@H]1CC[C@@H](C)C[C@H]1OC(=O)C[C@@H](NC(=O)[C@H](C)N)c1ccccc1 ; Cl] | 0.9136 | micro Molar |
| #15 | 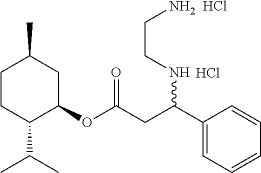 [SMILES: Cl ; CC(C)[C@@H]1CC[C@@H](C)C[C@H]1OC(=O)CC(NCCN)c1ccccc1 ; Cl] | 1.957 | micro Molar |
| #16 | 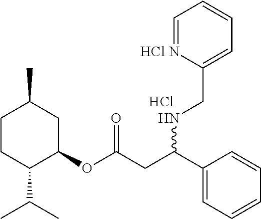 [SMILES: CC(C)[C@@H]1CC[C@@H](C)C[C@H]1OC(=O)CC(NCc1ccccn1)c1ccccc1 ; Cl ; Cl] | 2.073 | micro Molar |
| #17 | 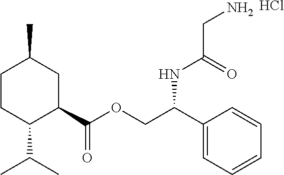 [SMILES: Cl ; CC(C)[C@@H]1CC[C@@H](C)C[C@H]1C(=O)OC[C@H](NC(=O)CN)c1ccccc1] | 2.26 | micro Molar |
| #18 | 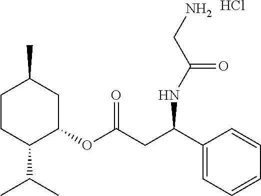 [SMILES: Cl ; CC(C)[C@@H]1CC[C@@H](C)C[C@@H]1OC(=O)C[C@@H](NC(=O)CN)c1ccccc1] | 2.951 | micro Molar |
| #19 | 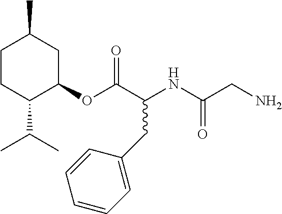 [SMILES: CC(C)[C@@H]1CC[C@@H](C)C[C@H]1OC(=O)C(Cc1ccccc1)NC(=O)CN] | 4.284 | micro Molar |
| #20 | 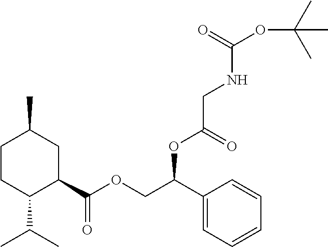 [SMILES: CC(C)[C@@H]1CC[C@@H](C)C[C@H]1C(=O)OC[C@@H](OC(=O)CNC(=O)OC(C)(C)C)c1ccccc1] | 4.473 | micro Molar |
| #21 | 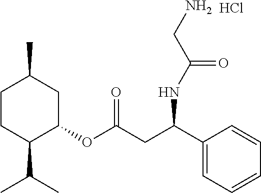 [SMILES: CC(C)[C@H]1CC[C@@H](C)C[C@@H]1OC(=O)C[C@@H](NC(=O)CN)c1ccccc1 ; Cl] | 4.958 | micro Molar |
| #22 | 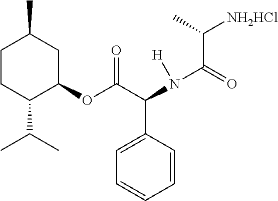 | 32.8 | micro Molar |
| #23 | 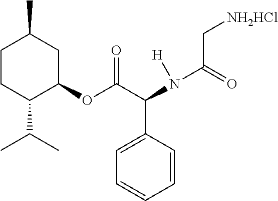 | 34.1 | micro Molar |
| #24 | 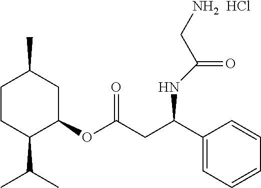 [SMILES: Cl ; CC(C)[C@H]1CC[C@@H](C)C[C@H]1OC(=O)C[C@@H](NC(=O)CN)c1ccccc1] | na | |
| #25 | 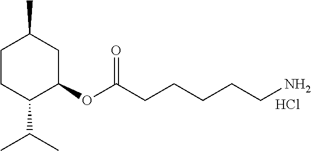 [SMILES: CC(C)[C@@H]1CC[C@@H](C)C[C@H]1OC(=O)CCCCCN ; Cl] | 9.5 | micro Molar |
| #26 | 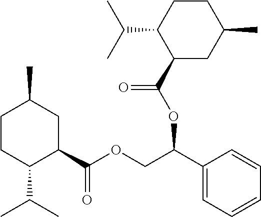 [SMILES: CC(C)[C@@H]1CC[C@@H](C)C[C@H]1C(=O)OC[C@@H](OC(=O)[C@@H]1C[C@H](C)CC[C@H]1C(C)C)c1ccccc1] | 139.4 | micro Molar |
| #27 | 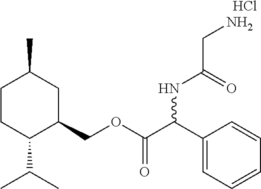 [SMILES: Cl ; CC(C)[C@@H]1CC[C@@H](C)C[C@H]1COC(=O)C(NC(=O)CN)c1ccccc1] | 144.6 | micro Molar |
| #28 | 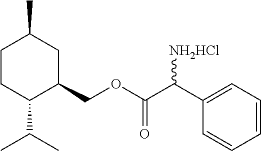 | na | |
| #29 | 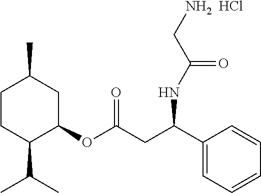 [SMILES: Cl ; CC(C)[C@H]1CC[C@@H](C)C[C@H]1OC(=O)C[C@@H](NC(=O)CN)c1ccccc1] | na | |
| #30 | 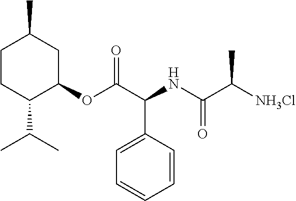 | 17 | micro Molar |
| #31 | 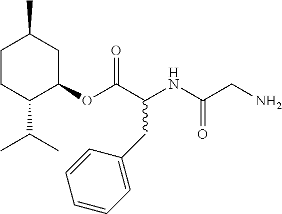 [SMILES: CC(C)[C@@H]1CC[C@@H](C)C[C@H]1OC(=O)C(Cc1ccccc1)NC(=O)CN] | 4.284 | micro Molar |
| #32 | 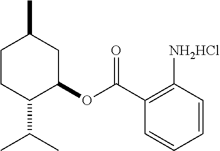 | na | |
| #33 | 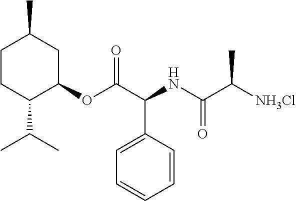 | 17 | micro Molar |
| #34 | 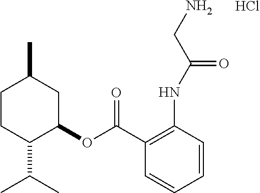 | 19 | micro Molar |

The EC50 values shown in EXAMPLE 1 provided examples of the unique sensates that were synthesized for their TRPM8 activity.

### Example 35

### Cooling Dentifrice Formulation

Dentifrices were prepared using conventional methods, such as the protocols described in U.S. Pat. No. 8,747,814, which contained no coolant (SAMPLE A—Crest Cavity Protection) or having a coolant from TABLE 1 (SAMPLE B), in a flavor (wintergreen) at 10 parts per million (ppm).

TABLE 2
Dentifrice formulations containing the compounds from TABLE 1

| Ingredient | A (Control) | B |
|---|---|---|
| FD&C Blue #1 Color Solution | 0.045% | 0.045% |
| Sodium Fluoride | 0.243% | 0.243% |
| CARBOMER 956 | 0.300% | 0.300% |
| Sodium Saccharin | 0.300% | 0.300% |
| Sodium Phosphate, Monobasic, Monohydrate | 0.419% | 0.419% |
| Titanium Dioxide | 0.525% | 0.525% |
| Carboxymethycellulose Sodium | 0.800% | 0.800% |
| Wintergreen Flavor | 1.000% | 1.000% |
| Coolant | 0% | — |
| Coolant Sample #1 (TABLE 1) | — | 0.001% |
| Tribasic Sodium Phosphate Dodecahydrate | 1.100% | 1.100% |
| Sodium Lauryl Sulfate 28% Solution | 4.000% | 4.000% |
| Silica, Dental Type, NF (Zeodent 119) | 15.000% | 15.000% |
| SORBITOL SOLUTION LRS USP | 54.673% | 54.673% |
| Water Purified, USP, PhEur, JP, JSCI | QS* | QS* |

*QS refers to the term quantum sufficit, meaning as much as suffices, where the remainder of the formula hole is filled with this substance.

Sensory evaluation studies of coolant activity were conducted using a methodology patterned after the techniques described in M. C. Meilgaard, et al., Sensory Evaluation Techniques, 4th Ed. (2007). Seven panelists brushed with a dentifrice from TABLE 2, having no coolant (SAMPLE A) or having a coolant from TABLE 1 (SAMPLE B), for two minutes, in a flavor (wintergreen) at 10 parts per million (ppm). After brush expectoration, panelists then rinsed their mouth with 15 ml of an aqueous rinse and expectorated. As shown in TABLE 3, panelists then evaluated cooling intensity, assigning a number between 0, which is no cooling sensation, to 90, which is a sensation as cold as ice.

TABLE 3
Cooling Rating (0-90) of Dentifrice over 8 hours

| Time | Avg 7 panelists of Toothpaste | Avg 7 panelists of Crest |
|---|---|---|
| (min) | with compound #1 | Cavity Protection |
| 0 | 33.57 | 15.00 |
| 15 | 32.71 | 6.67 |
| 30 | 26.43 | 2.83 |
| 60 | 35.71 | 0.17 |
| 120 | 40.00 | 0.00 |
| 180 | 36.43 | 0.00 |
| 240 | 34.29 | 0.00 |
| 300 | 30.71 | 0.00 |
| 360 | 30.71 | 0.00 |
| 420 | 23.57 | 0.00 |
| 480 | 20.71 | 0.00 |

### Example 36

### Shampoo Examples

TABLES 4A to 4C show antidandruff shampoo formulations containing compound #1 from TABLE 1. The samples were prepared by ordinary methods known in the art.

TABLE 4A

| Sample compositions | A | B | C | D | E | F | G | H | I | J |
|---|---|---|---|---|---|---|---|---|---|---|
| Sodium Laureth Sulfate | | | | | | | | | | |
| (SLE₃S) (1) | | | | | | | | | | |
| Sodium Laureth Sulfate | 10.5 | 10.5 | 10.5 | 10.5 | 10.5 | 10.5 | 10.5 | 10.5 | 10.5 | 12 |
| (SLE₁S) (2) | | | | | | | | | | |
| Sodium Lauryl Sulfate | 1.5 | 1.5 | 1.5 | 1.5 | 1.5 | 1.5 | 1.5 | 1.5 | 1.5 | |
| (SLS) (3) | | | | | | | | | | |
| Cocamidopropyl Betaine (4) | 1 | 1 | 1.25 | 1.25 | 1.25 | 1.25 | 1.25 | 1.25 | 1.25 | 1.5 |
| Cocamide MEA (5) | 1 | 1 | 1.5 | 1.5 | 1.5 | 1.5 | 1.5 | 1.5 | 1.5 | 1.5 |
| Glycol Distearate (6) | 1.5 | 1.5 | 1.5 | 1.5 | 1.5 | 1.5 | 1.5 | 1.5 | 1.5 | 1.5 |
| Zinc Pyrithione (7) | 1 | 1 | 1 | 1 | 1 | 1 | 1 | 1 | 1 | 1 |
| Zinc Carbonate (8) | 1.61 | 1.61 | 1.61 | 1.61 | 1.61 | 1.61 | 1.61 | 1.61 | 1.61 | 1.61 |
| Menthol (9) | 0 | 0 | 0 | 0 | 0 | 0.2 | 0.3 | 0.45 | 0.45 | 0.2 |
| Coolant Sample #1 or #2 | 0.001 | 0.045 | 0.001 | 0.045 | 0.045 | 0.001 | 0.045 | 0.01 | 0 | 0.001 |
| (TABLE 1) | | | | | | | | | | |
| Fragrance/Vanillin | 0.65 | 0.65 | 0.65 | 0.65 | 0 | 0.65 | 0.65 | 0.65 | 0.65 | 0.65 |
| Isobutyrate (11) | | | | | | | | | | |
| Glycerol (12) | | | | | | | | | | 1 |
| Guar | 0.3 | 0.3 | 0.3 | 0.3 | 0.3 | 0.3 | 0.3 | 0.3 | 0.3 | 0.3 |
| Hyrdroxypropyltrimonium | | | | | | | | | | |
| Chloride (LMW) (13) | | | | | | | | | | |
| Polyquaternium-10 | | | | | | | | | | 0.1 |
| (HMW) (14) | | | | | | | | | | |
| Polyquaternium 76 | 0.01 | 0.01 | 0.01 | 0.01 | 0.01 | 0.01 | 0.01 | 0.01 | 0.01 | |
| (AM:Triquat) (15) | | | | | | | | | | |
| Stearyl Alcohol (16) | | | | | | | | | | |
| Cetyl Alcohol (17) | | | | | | | | | | |
| Dimethicone (18) | 1.7 | 1.7 | 0.8 | 0.8 | 0.8 | 0.8 | 0.8 | 0.8 | 1.7 | 0.8 |
| Hydrochloric acid (19) | QS | QS | QS | QS | QS | QS | QS | QS | QS | QS |
| Preservative (20) | 0.05 | 0.05 | 0.05 | 0.05 | 0.05 | 0.05 | 0.05 | 0.05 | 0.05 | 0.05 |
| Sodium Chloride (21) | QS | QS | QS | QS | QS | QS | QS | QS | QS | QS |
| Sodium Xylene | QS | QS | QS | QS | QS | QS | QS | QS | QS | QS |
| Sulfonate (22) | | | | | | | | | | |
| Sodium Benzoate (23) | 0.27 | 0.27 | 0.27 | 0.27 | 0.27 | 0.27 | 0.27 | 0.27 | 0.27 | 0.27 |
| Water and Minors (QS | QS | QS | QS | QS | QS | QS | QS | QS | QS | QS |
| to 100%) (24) | | | | | | | | | | |

(1) Sodium Laureth-3 Sulfate from the Stepan Company
(2) Sodium Laureth-1 Sulfate from the Stepan Company
(3) Sodium Lauryl Sulfate from Stepan Company
(4) Amphosol HCA from Stepan Company
(5) Ninol COMF from Stepan Company
(6) EGDS from Golschmidt Chemical Company
(7) ZPT from Lonza
(8) Zinc Carbonate from Bruggeman Group
(9) Menthol from Kerry
10. Coolant Sample #1 or #2 (TABLE 1)
(11) Fragrance/Vanillin Isobutryate from Givuadan
(12) Glycerin from Procter & Gamble
(13) Jaguar C500 from Solvay with a M. Wt of 500,000 g/mol and charge density of 0.8 meq/g
(14) JR 30M from Dow with M. Wt of 2,000,000 g/mol with charge density of 1.3 meq/g
(15) Polyquaternium 76 from Solvay
(16) Stearyl Alcohol CO1895 from Procter and Gamble
(17) Cetyl Alcohol CO 1695 from Procter and Gamble
(18) Dimethicone Viscasil 330M from Momentive Performance Materials with a viscosity of 330,000 cSt (Centistokes)
(19) Hydrochloric acid from Mallinckrodt Baker Inc.
(20) Preservative Kathon CG from Akzo Nobel
(21) Sodium Chloride USP (food grade) Supplier Morton
(22) Sodium Xylene Sulfonate from Stepan Company
(23) Sodium Benzoate from Kalama Chemical
(24) Water from Misty Mountain Spring Water
*QS refers to the term quantum sufficit, meaning as much as suffices, where the remainder of the formula hole is filled with this substance.

TABLE 4B

| Sample compositions | K | L | M | N | O | P | Q | R | S | T |
|---|---|---|---|---|---|---|---|---|---|---|
| Sodium Laureth Sulfate (SLE₃S) (1) | | | | | | | 6 | 6 | 6 | 6 |
| Sodium Laureth Sulfate (SLE₁S) (2) | 12 | 12 | 12 | 12 | 12 | 12 | | | | |
| Sodium Lauryl Sulfate (SLS) (3) | | | | | | | 7 | 7 | 7 | 7 |
| Cocamidopropyl Betaine (4) | 1.5 | 1.5 | 1.5 | 1.5 | 1.5 | 1.5 | 1 | 1 | 1 | 1 |
| Cocamide MEA (5) | 1.5 | 1.5 | 1.5 | 1.5 | 1.5 | 1.5 | | | | |
| Glycol Distearate (6) | 1.5 | 1.5 | 1.5 | 1.5 | 1.5 | 1.5 | 1.5 | 1.5 | 1.5 | 1.5 |
| Zinc Pyrithione (7) | 1 | 1 | 1 | 1 | 1 | 1 | 1 | 1 | 1 | 1 |
| Zinc Carbonate (8) | 1.61 | 1.61 | 1.61 | 1.61 | 1.61 | 1.61 | 1.61 | 1.61 | 1.61 | 1.61 |
| Menthol (9) | 0.45 | 0 | 0 | 0 | 0.45 | 0 | 0 | 0.2 | 0 | 0 |
| Coolant Sample #1 or #2 (TABLE 1) | 0.01 | 0.045 | 0.02 | 0.001 | 0.01 | 0.045 | 0.001 | 0.02 | 0.045 | 0.045 |
| Fragrance/Vanillin Isobutyrate (11) | 0.65 | 0.65 | 0.65 | 0.65 | 0.65 | 0.65 | 0.65 | 0.65 | 0.65 | 0.65 |
| Glycerol (12) | 1 | 1 | 1 | 1 | 1 | 1 | | | | |
| Guar Hydroxypropyltrimonium | 0.3 | 0.3 | 0.3 | 0.3 | 0.3 | 0.3 | 0.23 | 0.23 | 0.23 | 0.23 |
| Chloride (LMW) (13) | | | | | | | | | | |
| Polyquaternium-10 | 0.1 | 0.1 | 0.1 | 0.1 | 0.1 | 0.1 | | | | |
| (HMW) (14) | | | | | | | | | | |
| Polyquaternium 76 (AM:Triquat) (15) | | | | | | | | | 0.01 | 0.01 |
| Stearyl Alcohol (16) | | | | 1.29 | 1.29 | 1.29 | | | | |
| Cetyl Alcohol (17) | | | | 0.71 | 0.71 | 0.71 | | | | |
| Dimethicone (18) | 0.8 | 0.8 | 0.8 | 0.8 | 0.8 | 0.8 | 0.8 | 0.8 | 0.8 | 1.7 |
| Hydrochloric acid (19) | QS | QS | QS | QS | QS | QS | QS | QS | QS | QS |
| Preservative (20) | 0.05 | 0.05 | 0.05 | 0.05 | 0.05 | 0.05 | 0.05 | 0.05 | 0.05 | 0.05 |
| Sodium Chloride (21) | QS | QS | QS | QS | QS | QS | QS | QS | QS | QS |
| Sodium Xylene Sulfonate (22) | QS | QS | QS | QS | QS | QS | QS | QS | QS | QS |
| Sodium Benzoate (23) | 0.27 | 0.27 | 0.27 | 0.27 | 0.27 | 0.27 | 0.27 | 0.27 | 0.27 | 0.27 |
| Water and Minors (QS to 100%) (24) | QS | QS | QS | QS | QS | QS | QS | QS | QS | QS |

1. Sodium Laureth-3 Sulfate from the Stepan Company
2. Sodium Laureth-1 Sulfate from the Stepan Company
3. Sodium Lauryl Sulfate from Stepan Company
4. Amphosol HCA from Stepan Company
5. Ninol COME from Stepan Company
6. EGDS from Golschmidt Chemical Company
7. ZPT from Lonza
8. Zinc Carbonate from Bruggeman Group
9. Menthol from Kerry
10. Coolant Sample #1 or #2 (TABLE 1)
11. Fragrance/Vanillin Isobutryate from Givuadan
12. Glycerin from Procter & Gamble
13. Jaguar C500 from Solvay with a M. Wt of 500,000 g/mol and charge density of 0.8meq/g
14. JR 30M from Dow with M.Wt of 2,000,000 g/mol with charge density of 1.3meq/g
15. Polyquaternium 76 from Solvay
16. Stearyl Alcohol C01895 from Procter and Gamble
17. Cetyl Alcohol CO 1695 from Procter and Gamble
18 Dimethicone Viscasil 330M from Momentive Performance Materials with a viscosity of 330,000 cSt (Centistokes)
19. Hydrochloric acid from Mallinckrodt Baker Inc.
20 Preservative Kathon CG from Akzo Nobel
21 Sodium Chloride USP (food grade) Supplier Morton
22. Sodium Xylene Sulfonate from Stepan Company
23. Sodium Benzoate from Kalama Chemical
24 Water from Misty Mountain Spring Water
*QS refers to the term quantum sufficit, meaning as much as suffices, where the remainder of the formula hole is filled with this substance.

TABLE 4C

| Example compositions | U | V | W | X |
|---|---|---|---|---|
| Sodium Laureth Sulfate | | | | |
| (SLE₃S) (1) | | | | |
| Sodium Laureth Sulfate | 12 | 14 | 12 | 14 |
| (SLE₁S) (2) | | | | |
| Sodium Lauryl Sulfate | | | | |
| (SLS) (3) | | | | |
| Cocamidopropyl Betaine (4) | 1.5 | 1.5 | 1.5 | 1.5 |
| Cocamide MEA (5) | 1.5 | 1.5 | 1.5 | 1.5 |
| Glycol Distearate (6) | 1.5 | 1.5 | 1.5 | 1.5 |
| Octopirox | 1 | 1 | 1 | 1 |
| Zinc Pyrithione (7) | 1 | 1.5 | | |
| Zinc Carbonate (8) | 1.61 | 1.61 | | |
| Menthol (9) | 0 | 0.45 | 0.2 | 0.45 |
| Coolant Sample #1 or #2 | 0.001 | 0.045 | 0.001 | 0.045 |
| (TABLE 1) | | | | |
| Fragrance/Vanillin | 0.65 | 0.65 | 0.65 | 0.65 |
| Isobutyrate (11) | | | | |
| Glycerol (12) | | | | |
| Guar | 0.3 | 0.3 | 0.4 | 0.4 |
| Hyrdroxypropyltrimonium | | | | |
| Chloride (LMW) (13) | | | | |
| Polyquaternium-10 | | 0.1 | | 0.1 |
| (HMW) (14) | | | | |
| Polyquaternium 76 | 0.01 | | 0.01 | |
| (AM:Triquat) (15) | | | | |
| Stearyl Alcohol (16) | | | | |
| Cetyl Alcohol (17) | | | | |
| Dimethicone (18) | 1.7 | 1.7 | 1.7 | 1.7 |
| Hydrochloric acid (19) | QS | QS | QS | QS |
| Preservative (20) | 0.05 | 0.05 | 0.05 | 0.05 |
| Sodium Chloride (21) | QS | QS | QS | QS |
| Sodium Xylene | QS | QS | QS | QS |
| Sulfonate (22) | | | | |
| Sodium Benzoate (23) | 0.27 | 0.27 | 0.27 | 0.27 |
| Water and Minors (QS | QS | QS | QS | QS |
| to 100%) (24) | | | | |

(1) Sodium Laureth-3 Sulfate from the Stepan Company
(2) Sodium Laureth-1 Sulfate from the Stepan Company
(3) Sodium Lauryl Sulfate from Stepan Company
(4) Amphosol HCA from Stepan Company
(5) Ninol COMF from Stepan Company
(6) EGDS from Golschmidt Chemical Company
(7) ZPT from Lonza
(8) Zinc Carbonate from Bruggeman Group
(9) Menthol from Kerry
(10) Coolant Sample #1 or #2 (TABLE 1)
(11) Fragrance/Vanillin Isobutryate from Givuadan
(12) Glycerin from Procter & Gamble
(13) Jaguar C500 from Solvay with a M. Wt of 500,000 g/mol and charge density of 0.8 meq/g
(14) JR 30M from Dow with M.Wt of 2,000,000 g/mol with charge density of 1.3 meq/g
(15) Polyquaternium 76 from Solvay
(16) Stearyl Alcohol CO1895 from Procter and Gamble
(17) Cetyl Alcohol CO 1695 from Procter and Gamble
(18) Dimethicone Viscasil 330M from Momentive Performance Materials with a viscosity of 330,000 cSt (Centistokes)
(19) Hydrochloric acid from Mallinckrodt Baker Inc.
(20) Preservative Kathon CG from Akzo Nobel
(21) Sodium Chloride USP (food grade) Supplier Morton
(22) Sodium Xylene Sulfonate from Stepan Company
(23) Sodium Benzoate from Kalama Chemical
(24 Water from Misty Mountain Spring Water Octopirox from Clariant
*QS refers to the term quantum sufficit, meaning as much as suffices, where the remainder of the formula hole is filled with this substance.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound comprising the following structure: 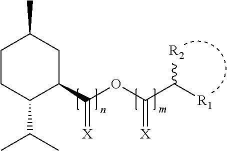
n=0 to 4; m=0 to 7; and wherein X is independently selected from O or H,H
R₁ and R₂ are independently selected from H, C, alkyl, aryl, substituted aryl, heteroalkyl, amino, amido, aminoalkyl, alkoxy; or when bound together, form part of an aromatic ring system; and including any acceptable salts or solvates thereof.

2. The compound of claim 1, wherein the compound activates at least one of TRPA1, TRPV1, or TRPM8.

3. A personal care composition comprising the compound of claim 1.

4. The personal care composition of claim 3 comprising an additional TRPM8 agonist.

5. The personal care composition of claim 4, wherein the additional TRPM8 agonists comprises at least one of Menthol; Menthyl Lactate; N-ethyl-ρ-menthan-3-carboxamide; N-ethoxycarbonylmethyl-ρ-menthan-3-carboxamide; N-(4-methoxyphenyl)-ρ-menthan-3-carboxamide; N-tert-butyl-ρ-menthan-3-carboxamide; N,2,3-trimethyl-2-isopropylbutanamide; N-(4-cyanomethylphenyl)-ρ-menthanecarboxamide; N-(4-sulfamoylphenyl)-ρ-menthanecarboxamide; N-(4-cyanophenyl)-ρ-menthanecarboxamide; N-(4-acetylphenyl)-ρ-menthanecarboxamide; N-(4-hydroxymethylphenyl)-ρ-menthanecarboxamide; N-(3-hydroxy-4-methoxyphenyl)-ρ-menthanecarboxamide; Isopulegol; and/or (−)-Menthoxypropane-1,2-diol.

6. The personal care composition of claim 3 and at least one of a TRPA1 agonist or TRPV1 agonist.

7. The personal care composition of claim 6, wherein the TRPA1 agonist is at least one of allyl isothiocyanate; menthol; peroxide; methyl salicylate; cinnamic aldehyde; benzyl alcohol; zinc salts; and/or vanillin isobutyrate.

8. The personal care composition of claim 6, wherein the TRPV1 agonist is at least one capsaicin; piperine; vanillyl butyl ether; vanillyl ethyl ether; menthol; peroxide; zinc salts; or an anti-histamine.

9. The compound of claim 1, wherein the compound at a concentration of about 5.2E-5% provides a greater activation of TRPM8 than WS5 at a concentration of about 30 mM.

10. The compound of claim 9, wherein the compound at a concentration of about 5.2E-5% provides a greater activation of TRPA1 than allyl isothiocyanate at a concentration of about 50 mM.

11. The compound of claim 10, wherein the compound at a concentration of about 5.2E-5% provides a greater activation of TRPV1 than capsaicin at a concentration of about 350 nM.

12. The personal care composition of claim 3 and at least one of a TRPA1 antagonist or TRPV1 antagonist.

13. The personal care composition of claim 12, wherein the TRPA1 antagonist is at least one of maltyl isobutyrate, tyramine, D-gluconic acid, lactic acid, pyegum bark extract, bayberry root, cinnamon bark oil; Phloretin; γ-Dodecalactone; vanillic acid; γ-Methyl Decalactone; trans, trans-2,4-Nonadienal; 4-Allyl-2,6-dimethoxyphenol; o-Methoxycinnamaldehyde; 4-Methyl-2-phenyl-2 Pentenal (mix of cis and trans); 2-Methoxy-4-propyl-phenol; Methyl 2-methoxy-benzoate; 6-Tetradecalactone; 1-Methyl-2-pyrole carboxaldehyde; 3,3,5-Trimethylcyclohexanol; N-(2-Hydroxyethyl) lactamide; 2-(3-Phenylpropyl) tetrahydrofuran; Anisyl Butyrate; Methyl-4-phenyl butyrate; 3-Heptyldihydro-5-methyl-2(3H)-furanone; 3-acetylsulfanylhexylacetate; 3-methyl-5-propyl-2-Cyclohexen-1-one; Isobornyl Isobutyrate; Bornyl Valerate; Citronellyl acetate; (2S,5S,6S)-6-) Hydroxy-dihydrotheaspirane; or trans-2-Hexenal.

14. The personal care composition of claim 12, wherein the TRPV1 antagonist is at least one of (−)-Bornyl Acetate; Hydroxycitronellal; Apritone; Methyl N,N-Dimethylanthranilate; 2-Ethoxy-3-ethylpyrazine; L-Piperiton; 4-Acetoxy-2,5-dimethyl-3(2H)-furanone; Tripropylamine; dihydrojasmone; 1-Methyl-2-pyrole carboxaldehyde; 3-Octyl Acetate; 2-Methylbutyl isovalerate; Jasminone; Piperonyl Isobutyrate; Phenoxyethyl Propionate; Vanillin Propylene Glycol Acetate; Octenyl Cyclopentanone; Guaiacwood Oil; or Tetrahydro-4-methyl-2-(2-methyl-1-propenyl)-2H pyran.

15. The compound of claim 1, wherein the structure comprises: 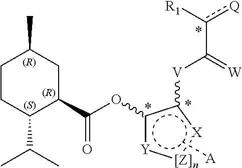
R₁ is selected from H, alkyl, amino alkyl, alkoxy
Q=H₂, O, OR₁, N(R₁)₂
V=NR₁, O
W=H₂, O
X, Y=independently selected from H, aryl, naphthyl for n=0
X, Y=aliphatic CH₂ or aromatic CH for n≥1 and Z is selected from aliphatic CH₂, aromatic CH, or heteroatom
A=lower alkoxy, lower alkylthio, aryl, substituted aryl or fused aryl
and stereochemistry is variable at the positions marked*; and including any acceptable salts or solvates thereof.

16. The compound of claim 15, wherein the structure comprises: 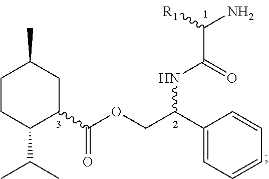
and including any acceptable salts or solvates thereof.

17. The compound of claim 16, wherein the variable stereochemistry at position #1, when R1 is an alkyl group, is either L or D; the stereochemistry position at position #2 is in the S-position; and the stereochemistry from the menthyl moiety at position #3 is in the L or in the neo-configuration.

18. A personal care composition comprising a compound comprising the following structure: 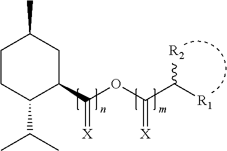
n=0 to 4; m=0 to 7; and wherein X is independently selected from O or H,H
R₁ and R₂ are independently selected from H, C, alkyl, aryl, substituted aryl, heteroalkyl, amino, amido, aminoalkyl, alkoxy; or when bound together, form part of an aromatic ring system; and including any acceptable salts or solvates thereof.

19. The personal care composition of claim 18, wherein the compound structure comprises: 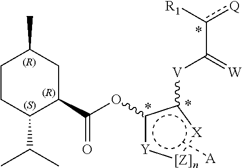
R₁ is selected from H, alkyl, amino alkyl, alkoxy
Q=H₂, O, OR₁, N(R₁)₂
V=NR₁, O
W=H₂, O
X, Y=independently selected from H, aryl, naphthyl for n=0
X, Y=aliphatic CH₂ or aromatic CH for n≥1 and Z is selected from aliphatic CH₂, aromatic CH, or heteroatom
A=lower alkoxy, lower alkylthio, aryl, substituted aryl or fused aryl
and stereochemistry is variable at the positions marked*; and including any acceptable salts or solvates thereof.

20. The personal care composition of claim 19, wherein the compound structure comprises: 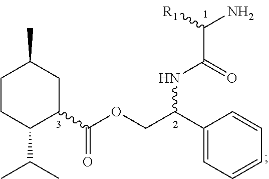
and including any acceptable salts or solvates thereof.

21. The personal care composition of claim 20, wherein the variable stereochemistry of the compound at position #1, when R1 is an alkyl group, is either L or D; the stereochemistry position at position #2 is in the S-position; and the stereochemistry from the menthyl moiety at position #3 is in the L or in the neo-configuration.

22. A method of preparing esters of menthol and menthol derivatives of Formula (I): 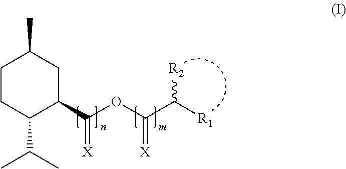
n=0 to 4; m=0 to 7; and wherein X is independently selected from O or H,H
R₁ and R₂ are independently selected from H, C, alkyl, aryl, substituted aryl, heteroalkyl, amino, amido, aminoalkyl, alkoxy; or when bound together, form part of an aromatic ring system.
comprising the steps of reacting an alcohol of Formula (II) in a coupling reaction: 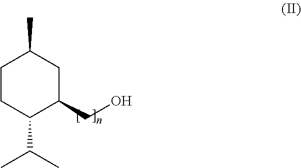
wherein n=0 to 4;
with an activated carboxylic acid derivative of Formula (III): 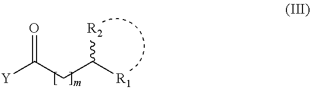
and wherein m=0 to 6 and Y is an activated leaving group.

23. A method of preparing menthylcarboxylic acid ester derivatives of Formula (I): 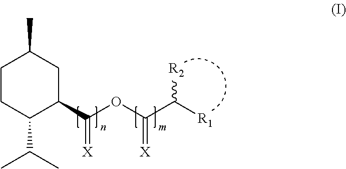
n=0 to 4; m=0 to 7; and wherein X is independently selected from O or H,H
R₁ and R₂ are independently selected from H, C, alkyl, aryl, substituted aryl, heteroalkyl, amino, amido, aminoalkyl, alkoxy; or when bound together, form part of an aromatic ring system.
comprising the steps of reacting a carboxylic acid derivative of Formula (II): 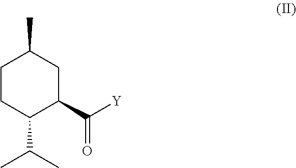
wherein Y is an activated leaving group;
in a coupling reaction with an alcohol derivative of Formula (III): 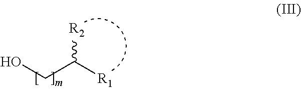
and wherein m=1 to 6.
